(19) United States
(12) Patent Application Publication
(10) Pub. No.: US 2026/0294906 A1
(43) Pub. Date: Oct. 1, 2026

(54) TREATING PSORIASIS USING A SMALL MOLECULE INHIBITOR OF TUMOR NECROSIS FACTOR ALPHA

(71) Applicant: SANOFI, Paris (FR)

(72) Inventors: Ohn CHOW, Conford, MA (US); Nassr NASSR, Hofheim am Taunus (DE); Mai Anh NGUYEN, Langen (DE); Laurent PERRIN, Montpellier (FR)

(21) Appl. No.: 19/477,466

(22) PCT Filed: Apr. 25, 2024
(86) PCT No.: PCT/EP2024/061394

§ 371 Date: Oct. 21, 2025

Related U.S. Application Data
(60) Provisional application No. 63/539,764, filed on Sep. 21, 2023.

(30) Foreign Application Priority Data
Apr. 26, 2023 (EP) 23315116.6

(51) Int. Cl. A61K 31/506; A61P 17/06

(52) U.S. Cl. CPC A61K 31/506; A61P 17/06

(57) ABSTRACT

Provided herein are treatments for psoriasis, wherein a human subject is administered a 400 mg daily dose of Compound 1 or a pharmaceutically acceptable salt thereof.

Provided herein is a treatment for psoriasis in which patients are administered a defined amount of a small molecule inhibitor of soluble tumor necrosis factor alpha. This is based on the results of a clinical trial which showed that treatment with the inhibitor over 28 days was effective, safe, and well-tolerated by participants with mild-to-moderate psoriasis.

### BACKGROUND

Tumor necrosis factor alpha (TNFα) is a cytokine with pleiotropic effects on both pathologic and homeostatic processes. TNFα is thought to have a key pathophysiological role in psoriasis (see, e.g., Mease, Ann Rheum Dis (2004) 63:755-758) and TNFα antagonists have become a cornerstone in modern management of psoriasis.

The TNFα is a member of the TNF superfamily of cytokines. It is produced by a variety of cell types, most notably, inflammatory cells. It is initially expressed as a transmembrane protein, also known as membrane-bound TNFα (mTNFα). At the cell surface, mTNFα may undergo cleavage by TNFα-converting enzyme to generate soluble TNFα (sTNFα). Structurally, both mTNFα and sTNFα exist as homotrimers which can bind three cognate receptors. This trimeric engagement is critical for receptor multimerization and subsequent signal transduction. sTNFα mainly signals via tumor necrosis factor receptor 1 (TNFR1), which is expressed on most cell types. In contrast, mTNFα mostly engages with tumor necrosis factor receptor 2 (TNFR2). TNFR2 is expressed primarily on neurons, endothelial cells, and immune cells, with particular enrichment on certain subpopulations of regulatory T-cells (Tregs).

Functionally, sTNFα and mTNFα play different roles in pathologic and homeostatic processes. sTNFα engagement of TNFR1 leads to activation of pro-inflammatory and pro-apoptotic pathways, which play pivotal roles in the effect of TNFα in certain autoimmune conditions. In contrast, mTNFα signaling contributes to a variety of homeostatic functions, including tissue regeneration and cell survival. From an inflammatory standpoint, mTNFα has been shown to enhance function and proliferation of specific subsets of suppressive Tregs. TNFR2 agonism has also been shown to promote death of autoreactive CD8+ T-cells. Of note, mTNFα mice, which express only a non-cleavable form of TNFα, have been generated to study the specific effects of mTNFα. In respiratory tuberculosis infection models, multiple studies have demonstrated reduced mortality and bacterial burden in mTNFα mice compared with TNFα knockout mice. Similar results have been demonstrated in Listeria monocytogenes, Mycobacterium bovis bacilli Calmette-Guérin, and Leishmania major murine infection models.

A naturally occurring, asymmetric trimeric form of sTNFα, which is normally a transient intermediate, has an impaired capacity to engage TNFR1 (see, e.g., O'Connell et al., Nat Commun (2019) 10:5795-5806). O'Connell et al. used analytical size exclusion to demonstrate the impaired capacity of sTNFα; they further demonstrated that UCB-9260, a molecule having a trisubstituted benzimidazole structure, stabilizes the asymmetric sTNFα trimer and impairs TNFR1 signaling in vitro. In that study, Jurkat cells were treated with sTNFα, sTNFα pre-incubated with UCB-9260, or sTNFα preincubated with etanercept. TNFR1 signaling, as measured by Western Blot analysis of kinases, receptor-interacting protein kinase 1 (RIP-1) ubiquitination and nuclear factor kappa B (NF-κB) phosphorylation, was impaired in both the etanercept- and UCB-9260-treated samples as compared with the samples treated with sTNFα alone.

The compound which is employed in the treatments of the present disclosure is (7R,14R)-11-[2-(1-aminocyclobutyl)pyrimidin-5-yl]-1-(difluoromethoxy)-6-methyl-6,7-dihydro-7,14-methanobenzimidazo[1,2-b][2,5]benzodiazocin-5 (14H)-one. The structure of this compound, Compound 1, is shown below and it has a pentacyclic core structure:

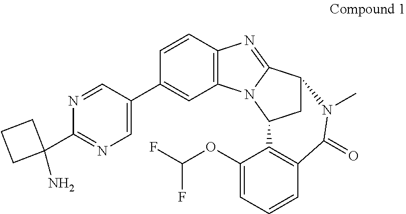 [SMILES: CN1C(=O)c2cccc(OC(F)F)c2[C@H]2C[C@@H]1c1nc3ccc(-c4cnc(C5(N)CCC5)nc4)cc3n12]

The synthesis of Compound 1 is described in international patent application No. PCT/EP2018/060489 (published as WO 2018/197503), which also describes the compound as belonging to a class of modulators of human TNFα activity which are useful for the treatment and/or prevention of various conditions, including a range of inflammatory and autoimmune disorders, neurological and neurodegenerative disorders, pain and nociceptive disorders, cardiovascular disorders, metabolic disorders, ocular disorders, and oncological disorders.

Psoriasis is a T cell-mediated disease with autoimmune characteristics modulated by genetic susceptibility along with environmental triggers. Inflammatory pathways marked with excessive production of cytokines interleukin-12 (IL-12) and interleukin-23 (IL-23), drive differentiation of pathogenic T cell responses resulting in TNF and interleukin-17 (IL-17) production. These cytokines are an integral part of the TNF/IL-23/IL-17 axis, which is responsible for maintaining inflammation in psoriatic skin (see, e.g., Bergen et al., Scand J Immunol. (2020) 92(4):e12946). Psoriasis is estimated to affect 1% to 3% of the world's population.

Historically, psoriasis has been treated using topical formulations (e.g., creams and ointments, which may have just an emollient effect, or which may include active agents such as steroids and/or vitamin D analogues), phototherapy, and systemic therapeutic treatments (e.g., treatment with oral inflammatory suppressors such as methotrexate, or immunosuppressants such as ciclosporin). More recently, biologic therapies which inhibit TNFα have been developed, such as etanercept (Enbrel®, which is authorized in the EU for the treatment of conditions including moderate and severe plaque psoriasis). Biologic therapies can be highly effective, but carry the risk of immunogenicity and development of neutralizing anti-drug antibodies, as well as side effects, some of which can be severe; they are also typically administered by injection and may require medical supervision or monitoring. There is, therefore, an acute need for new treatments for psoriasis which are effective, have an acceptable safety profile, and provide advantages in terms of ease of manufacture, storage, and suitability for oral administration.

The present disclosure provides results from a clinical trial in which Compound 1 was administered orally to patients with psoriasis. The results suggest that the treatment described herein is not only effective at reducing clinical symptoms, but is also effective at reducing the levels of inflammatory mediators implicated in the progression of the disease. There were no serious adverse effects identified during the course of the trial, no safety concerns based on vital signs, ECG or laboratory results, and no signal for risks on liver function tests. No indication for risks on QTc prolongation was observed. This is believed to be the first successful clinical trial of a small molecule inhibitor of TNFα in patients with psoriasis.

### SUMMARY

In a first aspect, the disclosure provides a compound for use in a method of treating psoriasis in a human subject, wherein the compound is (7R,14R)-11-[2-(1-aminocyclobutyl)pyrimidin-5-yl]-1-(difluoromethoxy)-6-methyl-6,7-dihydro-7,14-methanobenzimidazo[1,2-b][2,5]benzodiazocin-5(14H)-one (Compound 1):

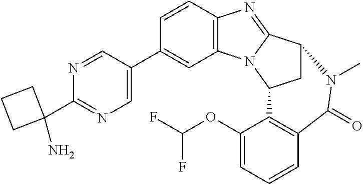 [SMILES: CN1C(=O)c2cccc(OC(F)F)c2[C@H]2C[C@@H]1c1nc3ccc(-c4cnc(C5(N)CCC5)nc4)cc3n12]

or a pharmaceutically acceptable salt thereof, wherein the subject has mild to moderate psoriasis, and wherein the method comprises administering to the subject a daily dose of about 400 mg of the compound (calculated as the free base).

In embodiments, the psoriasis is plaque psoriasis, e.g., chronic plaque psoriasis.

In embodiments, the severity of psoriasis in the subject is assessed using PASI scoring, and the subject has a total PASI score which is ≤16.

In embodiments, the subject has a total PASI score which is ≥10.

In embodiments, the subject has at least two lesions with TLS score ≥4 (excluding the scalp).

In embodiments, the compound is administered to the subject orally.

In embodiments, the compound is administered in the form of an oral pharmaceutical composition comprising Compound 1, or a pharmaceutically acceptable salt thereof, and at least one pharmaceutically acceptable excipient. In embodiments, the oral pharmaceutical composition is a tablet.

In embodiments, the method comprises administering to the subject a dose of about 200 mg of the compound (calculated as the free base) twice daily.

In a further aspect, the disclosure provides a method of treating psoriasis as defined hereinbefore, wherein a therapeutically effective amount of Compound 1 (or a pharmaceutically acceptable salt thereof) is administered to a human subject in need thereof, and wherein the therapeutically effective amount is a daily dose of about 400 mg (calculated as the free base).

In another aspect, the disclosure provides Compound 1, or a pharmaceutically acceptable salt thereof, for use in the manufacture of a medicament for treating mild to moderate psoriasis in accordance with a method as defined hereinbefore, wherein the medicament is adapted to provide a daily dose of about 400 mg of the compound (calculated as the free base).

Additional features and advantages of the compositions and methods disclosed herein will be apparent from the following detailed description.

### BRIEF DESCRIPTION OF THE DRAWINGS

FIG. 1 shows the mean (±SEM) change in TLS score from baseline over 2 and 4 weeks of treatment in human subjects with psoriasis. The solid line with filled circles is the placebo treatment arm, and the dashed line with open circles is the Compound 1 treatment arm.

FIG. 2 shows the mean (±SEM) change in total PASI score from baseline over 2 and 4 weeks of treatment in human subjects with psoriasis. The solid line with filled circles is the placebo treatment arm, and the dashed line with open circles is the Compound 1 treatment arm.

FIG. 3 shows the ratio from baseline over time for the serum levels of IL-17A, a systemic biomarker of psoriasis (data represent geometric mean (x/÷) geometric SEM). The light grey line (top) is the placebo treatment arm, and the dark grey line (bottom) is the Compound 1 treatment arm. * P<0.05; ** P=0.0001; *** P<0.0001.

FIG. 4 shows the ratio from baseline over time for the serum levels of IL-17F, a systemic biomarker of psoriasis (data represent geometric mean (x/÷) geometric SEM). The light grey line (top) is the placebo treatment arm, and the dark grey line (bottom) is the Compound 1 treatment arm. * P<0.05; ** P=0.0001; *** P<0.0001.

FIG. 5 shows the ratio from baseline over time for the serum levels of IL-22, a systemic biomarker of psoriasis (data represent geometric mean (x/÷) geometric SEM). The light grey line (top) is the placebo treatment arm, and the dark grey line (bottom) is the Compound 1 treatment arm. * P<0.05; ** P=0.0001; *** P<0.0001.

FIG. 6 shows the percentage of patients achieving a static Investigators Global Assessment (sIGA) score category (determined as described herein) at baseline, week 2, and week 4. Each sIGA category depicts paired bars, in which the proportion of patients from the placebo arm (N=12) is shown in the left-hand bar (grey), and the proportion of patients from the treatment arm (Compound 1, 200 mg BID; N=25) is shown in the right-hand bar (white).

### DEFINITIONS

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, exemplary methods, devices, and materials are now described. All technical and patent publications cited herein are incorporated herein by reference in their entirety.

The practice of the present disclosure will employ, unless otherwise indicated, conventional techniques of tissue culture, immunology, molecular biology, microbiology, cell biology, and recombinant DNA, which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, molecular weight, etc., including ranges, are approximations which are varied (+) or (−) by increments of, e.g., 0.1 or 1.0, where appropriate. It is to be understood, although not always explicitly stated, that all numerical designations are preceded by the term “about”, which is used to denote a conventional level of variability. For example, a numerical designation which is “about” a given value may vary by ±10% of said value; alternatively, the variation may be ±5%, ±2%, or ±10% of the value. For example, an amount of Compound 1 or a pharmaceutically acceptable salt thereof which is defined as being “about 400 mg (calculated as the free base)” may be an amount which is between 360 mg and 440 mg (calculated as the free base), i.e., 400 mg±10%; alternatively, the variation may be ±5%, ±2%, or ±10% of the value. It also is to be understood, although not always explicitly stated, that the reagents described herein are merely exemplary and that equivalents of such are known in the art.

As used in the specification and claims, the singular form “a”, “an”, and “the” include plural references unless the context clearly dictates otherwise. For example, the term “an inhibitor” includes a plurality of inhibitors, including mixtures thereof. Unless specifically stated or obvious from context, as used herein, the term “or” is understood to be inclusive. The term “including” is used herein to mean, and is used interchangeably with, the phrase “including but not limited to”.

As used herein, the term “comprising” or “comprises” is intended to mean that the compositions and methods include the recited elements, but do not exclude others. “Consisting essentially of” when used to define compositions and methods, shall mean excluding other elements of any essential significance for the stated purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure. Use of the term “comprising” herein is intended to encompass both “consisting essentially of” and “consisting of”.

A “subject”, “individual”, or “patient” is used interchangeably herein, and refers to a human.

“Administering” is defined herein as a means of providing an agent (e.g., active ingredient) or a composition containing the agent to a subject in a manner that results in the agent being inside the subject's body. Such an administration can be by any route including, without limitation, oral administration. Pharmaceutical preparations are, of course, given by forms suitable for each administration route. The compositions and methods of the present disclosure are typically directed towards enteral, e.g. oral, administration.

“Treating” or “treatment” of a disease includes: (1) inhibiting the disease, i.e. arresting or reducing the development of the disease or its clinical symptoms; and/or (2) relieving the disease, i.e. causing regression of the disease or its clinical symptoms. “Preventing” or “prevention” of a disease includes causing the clinical symptoms of the disease not to develop in a patient that may be predisposed to the disease but does not yet experience or display symptoms of the disease.

The term “suffering” as it relates to the term “treatment” refers to a patient or individual who has been diagnosed with the disease. The term “suffering” as it relates to the term “prevention” refers to a patient or individual who is predisposed to the disease. A patient may also be referred to being “at risk of suffering” from a disease because of a history of disease in their family lineage or because of the presence of genetic mutations associated with the disease. A patient at risk of a disease has not yet developed all or some of the characteristic pathologies of the disease.

An “effective amount” or “therapeutically effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, and the route of administration. It is understood, however, that specific dose levels of the therapeutic agents of the present disclosure for any particular subject depend upon a variety of factors including, for example, the activity of the specific compound employed, the age, body weight, general health, sex, and diet of the subject, the time of administration, the severity of the particular disorder being treated, and the form of administration. Typically, dosage-effect relationships from in vitro and/or in vivo tests initially can provide useful guidance on suitable doses for patient administration. In general, one will desire to administer an amount of the compound that is effective to achieve a serum level commensurate with the concentrations found to be effective in vitro.

Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to treat (e.g., improve) one or more symptoms associated with a disease or disorder described herein, ex vivo, in vitro, or in vivo.

As used herein, the term “pharmaceutically acceptable excipient” encompasses any of the standard pharmaceutical excipients, including carriers such as a phosphate buffered saline solution, water, and emulsions, such as an oil/water or water/oil emulsion, and various types of wetting agents. Pharmaceutical compositions also can include stabilizers and preservatives. For examples of carriers, stabilizers, and adjuvants, see Remington's Pharmaceutical Sciences (20th ed., Mack Publishing Co. 2000).

As used herein, the term “pharmaceutically acceptable salt” means a pharmaceutically acceptable acid addition salt or a pharmaceutically acceptable base addition salt of a currently disclosed compound that may be administered without any resultant substantial undesirable biological effect(s) or any resultant deleterious interaction(s) with any other component of a pharmaceutical composition in which it may be contained.

A mass quantity (e.g., a dosage amount) of Compound 1 referred to herein corresponds, unless expressly stated otherwise, to a mass of the compound calculated as the free base. For example, a 400 mg dose of compound refers to an amount of 400 mg of Compound 1 free base, or to an amount of a salt of Compound 1 which provides an equivalent molar quantity of Compound 1; this is referred to herein as an amount “calculated as the free base”.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The following abbreviations are used herein:
ADL activities of daily living
AE adverse event
AESI adverse event of special interest
ALT alanine aminotransferase
aPTT activated partial thromboplastin time
AST aspartate aminotransferase
BID twice a day (dosing)
BMI body mass index
BSA body surface area
CI confidence interval
CK creatine kinase
COVID-19 coronavirus disease 2019 (also SARS-CoV-2; severe acute respiratory syndrome coronavirus-2)
CPK creatine phosphokinase
(e)CRF (electronic) case report form
(hs)CRP (high sensitivity)C-reactive protein
CYP cytochrome P450
DBP diastolic blood pressure
DNA deoxyribonucleic acid
ECG electrocardiogram
ELISA enzyme-linked immunosorbent assay
EOS/EoS end of study
FSH follicle stimulating hormone
GGT gamma glutamyl transferase
(D-)HCG (beta-) human chorionic gonadotropin
HCV hepatitis C virus
hERG human ether-a-go-go related gene
HIV1/HIV2 human immunodeficiency virus 1/2
HR heart rate
HRT hormonal replacement therapy
ICF informed consent form
IL interleukin (e.g., IL-17A, IL-17F, IL-22)
IMP investigational medicinal product
INR international normalized ratio
LSm least square means
MMRM mixed model for repeated measures
NCI-CTCAE National Cancer Institute common terminology criteria for adverse events
NIMP non-investigational medicinal product
P-gp P-glycoprotein
PASI psoriasis area and severity index (the following abbreviations are used in connection with PASI scoring: head (h), upper extremities (u), trunk (t), lower extremities (l), numerical score (A), erythema (E), thickness/induration (I), and desquamation/scaling (D), such that “Eh” would denote erythema of the head, “It” would denote induration of the trunk, etc.)
PCR polymerase chain reaction
PCSA potentially clinically significant abnormalities
PCT procalcitonin
PD pharmacodynamic
PK pharmacokinetics
PT preferred term
QFT QuantiFERON-TB Gold test
QTcF corrected QT interval by Fridericia (QT interval=the time between the start of the QRS complex and the end of the T wave)
RNA nbonucleic acid
RT retention time
SAE serious adverse event
SBP systolic blood pressure
SD standard deviation
SE standard error
SEM standard error of the mean
sIGA static investigator's global assessment
SOC system organ class
SPF sun protection factor
TB tuberculosis
TEAE treatment-emergent adverse event
TLS(S) target lesion severity (score)
TNF(α) tumor necrosis factor (alpha)
mTNFα membrane-bound TNFα
sTNFα soluble TNFα
TNFR1/TNFR2 tumor necrosis factor receptor 1/2
ULN upper limit of normal
XPhos 2-dicyclohexylphosphino-2′,4′,6′-triisopropylbiphenyl

### DETAILED DESCRIPTION

Although specific embodiments of the present disclosure will now be described with reference to the preparations and schemes, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

The present disclosure describes a clinical study which has been carried out to assess the impact of Compound 1 on patients with mild and moderate psoriasis. Oral administration of 400 mg of Compound 1 per day was shown to be effective in improving clinical parameters in human patients, including Psoriasis Area and Severity Index (PASI) score, and Target Lesion Severity (TLS) score. Improvements in biomarkers for psoriasis were also observed, including serum levels of IL-17A, IL-17F and IL-22. No serious or severe adverse events were observed during the study, neither was any significant impact on cardiac parameters (e.g., QTcF interval) or liver parameters (e.g., AST, ALT, or bilirubin levels) observed.

Thus, a first aspect of the present disclosure provides a compound for use in a method of treating psoriasis in a human subject, wherein the compound is (7R,14R)-11-[2-(1-aminocyclobutyl)pyrimidin-5-yl]-1-(difluoromethoxy)-6-methyl-6,7-dihydro-7,14-methanobenzimidazo[1,2-b][2,5]benzodiazocin-5(14H)-one (i.e., Compound 1):

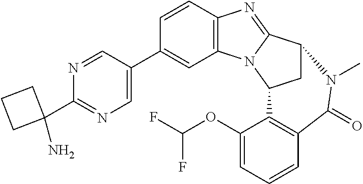 [SMILES: CN1C(=O)c2cccc(OC(F)F)c2[C@H]2C[C@@H]1c1nc3ccc(-c4cnc(C5(N)CCC5)nc4)cc3n12]

or a pharmaceutically acceptable salt thereof, wherein the subject has mild to moderate psoriasis, and wherein the method comprises administering to the subject a daily dose of about 400 mg of the compound (calculated as the free base).

A related aspect provides a method of treating psoriasis in a human subject in need thereof, wherein the subject has mild to moderate psoriasis, and wherein the method comprises administering a therapeutically effective amount of (7R,14R)-11-[2-(1-aminocyclobutyl)pyrimidin-5-yl]-1-(difluoromethoxy)-6-methyl-6,7-dihydro-7,14-methanobenzimidazo[1,2-b][2,5]benzodiazocin-5(14H)-one (i.e., Compound 1):

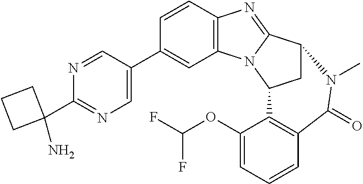 [SMILES: CN1C(=O)c2cccc(OC(F)F)c2[C@H]2C[C@@H]1c1nc3ccc(-c4cnc(C5(N)CCC5)nc4)cc3n12]

or a pharmaceutically acceptable salt thereof to the subject, wherein the therapeutically effective amount is a daily dose of about 400 mg (calculated as the free base).

Another related aspect provides a compound for use in the manufacture of a medicament for treating psoriasis in a human subject, wherein the compound is (7R,14R)-11-[2-(1-aminocyclobutyl)pyrimidin-5-yl]-1-(difluoromethoxy)-6-methyl-6,7-dihydro-7,14-methanobenzimidazo[1,2-b][2,5]benzodiazocin-5(14H)-one (i.e., Compound 1):

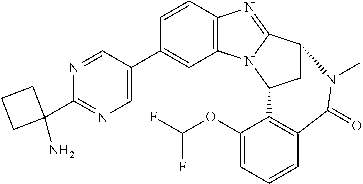 [SMILES: CN1C(=O)c2cccc(OC(F)F)c2[C@H]2C[C@@H]1c1nc3ccc(-c4cnc(C5(N)CCC5)nc4)cc3n12]

or a pharmaceutically acceptable salt thereof, wherein the subject has mild to moderate psoriasis, and wherein the medicament is adapted to provide the subject with a daily dose of about 400 mg of the compound (calculated as the free base).

In embodiments, the psoriasis is plaque psoriasis. In embodiments, the psoriasis is chronic plaque psoriasis.

In embodiments, the subject has mild psoriasis. Viewed from this aspect, the disclosure provides a compound for use in a method of treating mild psoriasis in a human subject, wherein the compound is Compound 1 or a pharmaceutically acceptable salt thereof, and wherein the method comprises administering to the subject a daily dose of about 400 mg of the compound (calculated as the free base). In other embodiments, the subject has moderate psoriasis. Viewed from this aspect, the disclosure provides a compound for use in a method of treating moderate psoriasis in a human subject, wherein the compound is Compound 1 or a pharmaceutically acceptable salt thereof, and wherein the method comprises administering to the subject a daily dose of about 400 mg of the compound (calculated as the free base).

In embodiments, the severity of psoriasis in the subject is assessed using PASI scoring, e.g. as described herein (see also, e.g., the EMA Guidelines on clinical investigation of medicinal products indicated for the treatment of psoriasis, Nov. 18, 2004). The subject may be classified as having mild to moderate psoriasis by virtue of having a total PASI score which is ≤16. Thus, in one embodiment the subject has a total PASI score which is ≤16. The subject may be classified as having mild psoriasis by virtue of having a total PASI score which is <10. Thus, in one embodiment the subject has a total PASI score which is <10. The subject may be classified as having moderate psoriasis if they have a total PASI score which is ≥10. Thus, in one embodiment the subject has a total PASI score which is ≥10.

Viewed from this latter aspect, the disclosure provides a compound for use in a method of treating psoriasis in a human subject, wherein the compound is Compound 1 or a pharmaceutically acceptable salt thereof, wherein the subject has a total PASI score of ≥10, and wherein the method comprises administering to the subject a daily dose of about 400 mg of the compound (calculated as the free base). In embodiments, the subject has a total PASI score which is ≥10 and ≤16.

In another aspect, the disclosure provides a compound for use in a method of treating psoriasis in a human subject, wherein the compound is Compound 1 or a pharmaceutically acceptable salt thereof, wherein the subject has a total PASI score of <10, and wherein the method comprises administering to the subject a daily dose of about 400 mg of the compound (calculated as the free base).

In embodiments, the treatment results in a reduction in total PASI score (e.g., as compared to baseline, which may be assessed immediately before treatment commences). In embodiments, the reduction is a reduction of at least 1 point, e.g. a reduction of about 1.5 points. In other embodiments, the reduction is a reduction of at least 2 points, e.g., a reduction of about 3 points. In embodiments, the reduction in total PASI score is a reduction of at least 15%, e.g. a reduction of at least 25% or at least 35%. In embodiments, the reduction is effected over a period of at least 2 weeks or at least 4 weeks. In embodiments, the treatment results in a reduction in total PASI score of about 1.5 points over a period of about 2 weeks.

In embodiments, the treatment results in a reduction in total PASI score of about 17% over a period of about 2 weeks. In embodiments, the treatment results in a reduction in total PASI score of about 3 points over a period of about 4 weeks. In embodiments, the treatment results in a reduction in total PASI score of about 35% over a period of about 4 weeks. In embodiments, the subject has a total PASI score of <10 and the treatment results in a reduction in total PASI score of about 21% over a period of about 2 weeks, and/or a reduction in total PASI score of about 34% over a period of about 4 weeks. In embodiments, the subject has a total PASI score of ≥10 and the treatment results in a reduction in total PASI score of about 9% over a period of about 2 weeks and/or a reduction in total PASI score of about 33% over a period of about 4 weeks.

In embodiments, the presence (or severity) of psoriasis in the subject is assessed using TLS scoring, e.g. as described herein (see also, e.g., Fredriksson et al., Dermatologica (1978) 157(4):238-244; as well as Czamowicki et al., J Am Acad Dermatol. (2014) 71(5):954-959.e1). In embodiments, the subject has at least two lesions with TLS score ≥4 (excluding the scalp). In embodiments, the subject has at least two lesions with TLS score ≥4 (excluding the scalp) and also has a total PASI score of ≤16.

In embodiments, the treatment results in a reduction in TLS score of one or more lesions (e.g., as compared to baseline, which may be assessed immediately before treatment commences). In embodiments, the TLS score is calculated as the average score from 2 lesions. In embodiments, the reduction is a reduction of at least 1 point. In embodiments, the reduction is a reduction of about 1 point. In other embodiments, the reduction is a reduction of at least 1.5 points, e.g., a reduction of about 2.5 points. In embodiments, the reduction is a reduction of at least 15%, e.g., a reduction of at least 35%. In embodiments, the reduction is effected over a period of at least 2 weeks or at least 4 weeks. In embodiments, the treatment results in a reduction in TLS score of about 1 point over a period of about 2 weeks. In embodiments, the treatment results in a reduction in TLS score of about 17% over a period of about 2 weeks. In embodiments, the treatment results in a reduction in TLS score of about 2.5 points over a period of about 4 weeks. In embodiments, the treatment results in a reduction in TLS score of about 38% over a period of about 4 weeks. In embodiments, the subject has a total PASI score of <10 and the treatment results in a reduction in TLS score of about 21% over a period of about 2 weeks, and/or a reduction in TLS score of about 37% over a period of about 4 weeks. In embodiments, the subject has a total PASI score of ≥10 and the treatment results in a reduction in TLS score of about 11% over a period of about 2 weeks and/or a reduction in TLS score of about 40% over a period of about 4 weeks.

In embodiments, the presence (or severity) of psoriasis in the subject is assessed using sIGA scoring, e.g. as described herein (see also, e.g., Langley et al., J Dermatolog. Treat. (2015) 26(1):23-31). The subject may be classified as having mild to moderate psoriasis by virtue of having a mean sIGA score (e.g., the mean average calculated from the scoring for erythema, induration, and scaling as described below) which is less than 4.5. Thus, in one embodiment the subject has a mean sIGA score which is >0.0 and which is <4.5, e.g. a mean sIGA score which is ≥1.5 and which is <3.5. In embodiments, the subject has a mean sIGA score which is >0.0 and which is <2.5, e.g. a mean sIGA score which is ≥1.5 and which is <2.5 (e.g., the subject is classified as having mild psoriasis). In embodiments, the subject has a mean sIGA score which is ≥2.5 and which is <4.5, e.g. a mean sIGA score which is ≥2.5 and which is <3.5 (e.g., the subject is classified as having moderate psoriasis). The sIGA scoring can also be used alongside other assessment measures, e.g. those described herein. Thus, in embodiments the subject has a mean sIGA score which is <4.5, and has: (i) at least two lesions with TLS score ≥4 (excluding the scalp); (ii) a total PASI score of ≤16; or (iii) at least two lesions with TLS score ≥4 (excluding the scalp) and a total PASI score of ≤16. In embodiments, the subject has a mean sIGA score which is <3.5, e.g. a mean sIGA score which is <2.5. The subject may be classified as having mild to moderate psoriasis on the basis of an overall sIGA score (e.g., a sIGA score translated as described below into an integer from 0 to 5). Thus, in one embodiment the subject has an overall sIGA score of 1, 2, 3, or 4. In embodiments the subject has an overall sIGA score of 1 or 2, e.g. an overall sIGA score of 2 (e.g., the subject is classified as having mild psoriasis). In embodiments, the subject has an overall sIGA score of 3 or 4, e.g. an overall sIGA score of 3 (e.g., the subject is classified as having moderate psoriasis).

In embodiments, the treatment results in a reduction in mean sIGA score (e.g., as compared to baseline). In embodiments, the reduction is effected over a period of at least about 2 weeks or at least about 4 weeks. In embodiments, the treatment results in a reduction in mean sIGA score of at least about 0.3, e.g. a reduction of at least about 0.6, 1.0, 1.3, or 1.6. In embodiments, the treatment results in a reduction in mean sIGA score of up to about 2.0, e.g. a reduction of up to about 1.7, 1.4, or 1.0. In embodiments, the reduction is a reduction of from about 0.3 to about 1.7. In embodiments, the treatment results in a reduction in mean sIGA score of from about 0.3 to about 1.7, e.g. from about 0.6 to about 1.4, such as about 1.0, over a period of about 2 weeks. In embodiments, the treatment results in a reduction in mean sIGA score of from about 0.3 to about 1.7, e.g. from about 0.6 to about 1.4, such as about 1.0, over a period of about 4 weeks. In embodiments, the subject has a mean sIGA score of less than 3.5 following treatment, e.g. mean sIGA score of less than about 2.5 or 1.5 following treatment. In embodiments, the subject has a mean sIGA score of from 0 to about 2.5, e.g. from 0 to about 1.5, following treatment over a period of at least about 2 weeks or at least about 4 weeks.

In embodiments, the treatment results in a reduction in overall sIGA score (e.g., as compared to baseline). In embodiments, the reduction is effected over a period of at least 2 weeks or at least 4 weeks. In embodiments, the treatment results in a reduction in overall sIGA score of at least 1 point, e.g. a reduction in overall sIGA score of 1 point or more. In embodiments, the reduction is a reduction of 1 point. In embodiments, the treatment results in a reduction in overall sIGA score of 1 point over a period of about 2 weeks. In embodiments, the treatment results in a reduction in overall sIGA score of 1 point over a period of about 4 weeks. In embodiments, the subject has an overall sIGA score of ≥3 following treatment, e.g. an overall sIGA score of ≥2 or ≥1 following treatment. In embodiments, the subject has an overall sIGA score of 1 or 2 following treatment over a period of at least about 2 weeks. In embodiments, the subject has an overall sIGA score of 1 or 2 following treatment over a period of at least about 4 weeks. In embodiments, the subject has an overall sIGA score of 1 following treatment over a period of at least 4 weeks.

In embodiments, the treatment results in a reduction in the level of one or more biomarkers of psoriasis in the subject, e.g., a reduction in the level of IL-17A, IL-17F, and/or IL-22 in the blood (e.g., serum) of the subject. In embodiments, the treatment results in a reduction in the level of IL-17A, e.g., a reduction in the level of IL-17A in the blood (e.g., serum) of the subject. In embodiments, the treatment results in a reduction in the serum level of IL-17A of at least about 0.05 pg/mL (e.g., as compared to baseline, which may be assessed immediately before treatment commences), e.g., a reduction of at least about 0.15 pg/mL. In embodiments, the treatment results in a reduction in the serum level of IL-17A of about 0.06 pg/mL at 2 weeks. In embodiments, the treatment results in a reduction in the serum level of IL-17A of about 0.18 pg/mL at 4 weeks. In embodiments, the treatment results in a reduction in the serum level of IL-17A of at least about 10% (e.g., as compared to baseline, which may be assessed immediately before treatment commences), e.g., at least about 25%. In embodiments, the treatment results in a reduction in the serum level of IL-17A of about 8% at 2 weeks. In embodiments, the treatment results in a reduction in the serum level of IL-17A of about 28% at 4 weeks. In embodiments, the treatment results in a reduction in the level of IL-17F, e.g., a reduction in the level of IL-17F in the blood (e.g., serum) of the subject. In embodiments, the treatment results in a reduction in the serum level of IL-17F of at least about 1.0 pg/mL (e.g., as compared to baseline, which may be assessed immediately before treatment commences), e.g., a reduction of at least about 1.2 pg/mL. In embodiments, the treatment results in a reduction in the serum level of IL-17F of about 1.0 pg/mL at 2 weeks. In embodiments, the treatment results in a reduction in the serum level of IL-17F of about 1.3 pg/mL at 4 weeks. In embodiments, the treatment results in a reduction in the serum level of IL-17F of at least about 35% (e.g., as compared to baseline, which may be assessed immediately before treatment commences), e.g., at least about 50%. In embodiments, the treatment results in a reduction in the serum level of IL-17F of about 39% at 2 weeks. In embodiments, the treatment results in a reduction in the serum level of IL-17F of about 52% at 4 weeks. In embodiments, the treatment results in a reduction in the level of IL-22, e.g., a reduction in the level of IL-22 in the blood (e.g., serum) of the subject. In embodiments, the treatment results in a reduction in the serum level of IL-22 of at least about 1.0 pg/mL (e.g., as compared to baseline, which may be assessed immediately before treatment commences), e.g., a reduction of at least about 1.5 pg/mL. In embodiments, the treatment results in a reduction in the serum level of IL-22 of about 1.2 pg/mL at 2 weeks. In embodiments, the treatment results in a reduction in the serum level of IL-22 of about 1.6 pg/mL at 4 weeks. In embodiments, the treatment results in a reduction in the serum level of IL-22 of at least about 30% (e.g., as compared to baseline, which may be assessed immediately before treatment commences), e.g., at least about 40%. In embodiments, the treatment results in a reduction in the serum level of IL-22 of about 34% at 2 weeks. In embodiments, the treatment results in a reduction in the serum level of IL-22 of about 43% at 4 weeks.

In embodiments, the subject at the outset of treatment satisfies one or more (e.g., all) criteria selected from: (a) male or female (e.g., male) and aged between 18 and 65 years, inclusive; (b) confirmed diagnosis of chronic plaque-type psoriasis, diagnosed at least 6 months prior to screening with mild to moderate severity, defined as PASI≤16; (c) at least two lesions with TLS score ≥4 (excluding the scalp); (d) in good health (except for psoriasis), e.g. as assessed by a clinician based on medical history, physical examination, vital signs, ECG, clinical laboratories, and urinalysis; (e) female and postmenopausal (e.g., no menses for 12 months without an alternative medical cause—a high FSH level in the postmenopausal range is used to confirm a postmenopausal state in women not using hormonal contraception or HRT) or sterilized (e.g., post-bilateral surgical oophorectomy not linked to a history of cancer); (f) laboratory parameters within the normal range, e.g., hepatic enzymes (ALT, AST) and GGT not exceeding ULN and total bilirubin value up to 1.5 ULN if associated with normal conjugated bilirubin value; (g) normal vital signs after at least 10 minutes resting in supine position at screening, e.g., 90 mmHg≤systolic blood pressure (SBP)≤140 mmHg, 45 mmHg≤diastolic blood pressure (DBP)≤90 mmHg, and 50 beats per minute (bpm)≤heart rate (HR)≤100 bpm; (h) standard 12-lead ECG parameters after 10 minutes resting in supine position, e.g., in the following ranges PR<220 ms, QRS<120 ms, QTcF≤450 ms, 50 bpm<HR<100 bpm and normal ECG tracing; (i) BMI between 18.0 and 35.0 kg/m², inclusive (body weight not under 50.0 kg); and (j) male and using contraception and agreeing not to donate sperm from the start of treatment up to 2.5 months after the last dosing.

In embodiments, the subject at the outset of treatment fails to satisfy any (e.g. all) of the criteria selected from: (a) pre-existing signs of skin atrophy, telangiectasia or striae in the affected area; (b) current evidence of non-plaque forms of psoriasis (e.g., erythrodermic, guttate or pustular) or psoriatic arthritis; (c) current evidence or suspicion of drug-induced psoriasis (e.g., new onset or exacerbation of psoriasis from beta blockers, calcium channel blockers, or lithium); (d) presence or history of drug hypersensitivity, or allergic disease diagnosed and treated by a physician; (e) opportunistic infections within 6 months of the intended treatment start date; (f) any previous gastrointestinal surgery or recent (within 3 months prior to intended treatment start date) history of gastrointestinal disease that could impact the absorption of Compound 1; (g) history of drug or alcohol abuse within the 12 months prior to intended treatment start date; (h) evidence of any clinically significant, severe or unstable, acute or chronically progressive, uncontrolled infection or medical condition (including an ongoing biological proven SARS-CoV-2 infection and recurrent infection) or any condition that may affect participant safety (e.g., not being adequately vaccinated against a SARS-CoV-2); (i) blood donation (any volume) within 2 months before intended treatment start date; (j) known allergy to local anaesthetics; (k) use of systemic immunosuppressants within 4 weeks of intended treatment start date; (1) use within 4 weeks prior to intended treatment start date of any systemic non-biologic psoriasis therapy (including, but not limited to psoralens and ultraviolet A therapy, cyclosporine, methotrexate, azathioprine, corticosteroids, apremilast, tofacitinib, oral retinoids, mycophenolate mofetil, sirolimus), or phototherapy (including ultraviolet B or self-treatment with tanning beds or therapeutic sunbathing), or topical psoriasis therapy with psoralens; (m) use of topical corticosteroid preparations (except hydrocortisone 1%), topical calcineurin inhibitors, or other topical preparations with immunomodulatory properties within 2 weeks prior to intended treatment start date; (n) prior use of any biologicals for treatment of psoriasis; (o) receipt of any live vaccination within 3 months, any initial non-live vaccination within 30 days, or non-live booster vaccination within 14 days of intended treatment start date; (p) use of any anticoagulants within 3 months of intended treatment start date, or of acetylsalicylic acid within 2 weeks prior to intended treatment start date; (q) use of sensitive P-gp, CYP3A4, CYP1A1 or CYP1A2 substrates, respectively, with a narrow therapeutic window or high-risk indication; (r) use of strong or moderate inducers or inhibitors of CYP3A4 or P-gp within 30 days or 5 half-lives from intended treatment start date, whichever is longer (this also includes the consumption of grapefruit, grapefruit juice, or grapefruit containing products within 72 hours of intended treatment start date; (s) use of strong or moderate inducers of CYP1A1/CYP1A2 within 30 days or 5 half-lives from intended treatment start date, whichever is longer (this also includes excessive consumption of beverages containing xanthine bases, e.g., more than 4 cups or glasses per day); (t) enrolment or participation in any clinical study involving an IMP or in any other type of medical research and still being in the exclusion period according to applicable regulations; (u) history of tuberculosis and/or a positive QuantiFERON-TB Gold test (QFT); (v) positive for hepatitis B surface antigen and/or hepatitis B core antibody, or positive for anti-human immunodeficiency virus 1 and 2 antibodies (anti-HIV1 and anti-HIV2 Ab); (w) hepatitis C antibody positive, unless successfully treated or, if never treated, then negative for hepatitis C virus (HCV) RNA by PCR; (x) positive result on urine drug screen (e.g., for amphetamines/methamphetamines, barbiturates, benzodiazepines, cannabinoids, cocaine, or opiates); (y) procalcitonin (PCT) higher than 0.5 ng/ml (equivalent to higher than 0.5 μg/L) with signs and symptoms of clinical evident infection; and (z) positive alcohol test.

The present disclosure contemplates salt forms of Compound 1, e.g., Compound 1 in the form of a pharmaceutically acceptable salt. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. In embodiments, the compound is a pharmaceutically acceptable salt of Compound 1. In embodiments, the compound is a pharmaceutically acceptable salt of Compound 1 and the method comprises administering to the subject a daily dose of the salt which is equivalent to 400 mg of the free base, e.g. equivalent to 200 mg of the free base BID. In other embodiments, the compound is Compound 1 free base. In embodiments, the compound is Compound 1 free base and the method comprises administering to the subject a daily dose of 400 mg, e.g. 200 mg BID.

In embodiments, the compound is administered to the subject orally. In embodiments, the compound is administered in the form of an oral pharmaceutical composition (e.g., dosage form) comprising Compound 1, or a pharmaceutically acceptable salt thereof, and at least one pharmaceutically acceptable excipient. The pharmaceutically acceptable excipient can be any such excipient known in the art including those described in, for example, Remington's Pharmaceutical Sciences, Mack Publishing Co. (A. R. Gennaro edit. 1985). Pharmaceutical compositions of the compound may be prepared by conventional means known in the art including, for example, mixing with one or more pharmaceutically acceptable excipient.

For oral administration, the pharmaceutical composition may take the form of, e.g., tablets, lozenges or capsules prepared by conventional means with pharmaceutically acceptable excipients such as binding agents (e.g., pregelatinised maize starch, polyvinylpyrrolidone, or hydroxypropyl methyl cellulose); fillers (e.g., lactose, microcrystalline cellulose, or calcium hydrogenphosphate); lubricants (e.g., magnesium stearate, talc, or silica); disintegrants (e.g., potato starch, or sodium glycollate); or wetting agents (e.g., sodium lauryl sulphate). The tablets may be coated by methods well known in the art. In embodiments, the oral pharmaceutical composition is a tablet. Preparations for oral administration may be suitably formulated to give controlled release of the active compound, e.g., using formulations of the type known in the art.

In embodiments, the treatment comprises administering to the subject a dose of about 200 mg of the compound (calculated as the free base) twice daily, e.g. 200 mg BID of Compound 1. In embodiments, the compound is administered in the form of tablets, e.g. tablets comprising 50 mg of the compound (calculated as the free base). In embodiments, the treatment comprises administering four tablets, each of which contains about 50 mg of the compound (calculated as the free base), twice a day, for a total daily dose of about 400 mg.

In embodiments, the treatment comprises administering the compound or pharmaceutically acceptable salt thereof to the subject with food, e.g., before, during, or immediately after a meal. In embodiments, the treatment comprises administering the compound or pharmaceutically acceptable salt thereof to the subject without regard to meals.

Thus, in one aspect, the present disclosure also provides a pharmaceutical composition comprising an effective amount of Compound 1, or a pharmaceutically acceptable salt thereof, and at least one pharmaceutically acceptable excipient. In embodiments, the pharmaceutical composition is adapted to provide a daily dose of about 400 mg of Compound 1 or a pharmaceutically acceptable salt thereof (calculated as the free base). In embodiments, the pharmaceutical composition is adapted to provide a twice daily dose of about 200 mg of Compound 1 or a pharmaceutically acceptable salt thereof (calculated as the free base). In embodiments, the pharmaceutical composition provides the Compound 1 or the pharmaceutically acceptable salt thereof in one or more (e.g., 1, 2, or 4) unit dosages. In embodiments, the pharmaceutical composition provides a unit dosage of about 200 mg of Compound 1 or a pharmaceutically acceptable salt thereof (calculated as the free base), e.g., for administration of two unit dosages per day. In embodiments, the pharmaceutical composition provides a unit dosage of about 50 mg of Compound 1 or a pharmaceutically acceptable salt thereof (calculated as the free base), e.g., for administration of eight unit dosages per day, e.g., for administration of four unit dosages twice daily.

The present disclosure provides Compound 1, or a pharmaceutically acceptable salt thereof, for use in methods of treating psoriasis. It will be appreciated that the disclosure also provides methods of treating psoriasis as described herein, in which a therapeutically effective amount of Compound 1, or a pharmaceutically acceptable salt thereof, as described herein is administered to a human subject in need thereof. It will also be appreciated that the disclosure further provides Compound 1 (or a pharmaceutically acceptable salt thereof) for use in the manufacture of a medicament for treating psoriasis in accordance with the methods disclosed herein.

Having been generally described herein, the follow non-limiting examples are provided to further illustrate the disclosure.

### EXAMPLES

### Example 1: Synthesis of (7R,14R)-11-[2-(1-Aminocyclobutyl)pyrimidin-5-yl]-1-(difluoromethoxy)-6-methyl-6,7-dihydro-7,14-methanobenzimidazo[1,2-b][2,5]benzodiazocin-5(14H)-one (Compound 1)

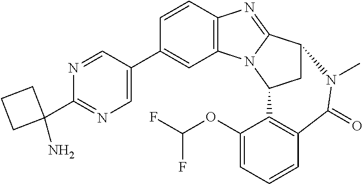 [SMILES: CN1C(=O)c2cccc(OC(F)F)c2[C@H]2C[C@@H]1c1nc3ccc(-c4cnc(C5(N)CCC5)nc4)cc3n12]

Compound 1 was prepared according to Example 6 of international patent application No. PCT/EP2018/060489 (published as WO 2018/197503). In brief, in a first step, N-(cyclobutylidene)-2-methylpropane-2-sulfinamide (Intermediate 13) was prepared by reacting cyclobutanone with 2-methyl-2-propanesulfinamide followed by titanium(IV) isopropoxide and purified by flash chromatography. In a second step, 5-bromo-2-iodopyrimidine was cooled and treated with n-butyllithium followed by Intermediate 13 to afford N-[1-(5-bromopyrimidin-2-yl)cyclobutyl]-2-methylpropane-2-sulfinamide (Intermediate 14), which was purified by flash chromatography. In a third step, Intermediate 14 was treated with MeOH/HCl followed by isopropyl ether/MeOH to afford 1-(5-bromopyrimidin-2-yl)cyclobutanamine hydrochloride (Intermediate 15). In a fourth step, Intermediate 15 was treated with di-tert-butyl dicarbonate and triethylamine to afford tert-butyl N-[1-(5-bromopyrimidin-2-yl)cyclobutyl]carbamate (Intermediate 16). In a fifth step, (7R,14R)-11-chloro-1-(difluoromethoxy)-6,7-dihydro-7,14-methanobenzimidazo[1,2-b][2,5]benzodiazocin-5(14H)-one (prepared according to Example 11 of WO 2016/050975) was treated with potassium bis(trimethylsilyl)amide followed by iodomethane to afford (7R,14R)-11-chloro-1-(difluoromethoxy)-6-methyl-6,7-dihydro-7,14-methano-benzimidazo[1,2-b][2,5]benzodiazocin-5(14H)-one (Intermediate 3) which was purified by flash chromatography. In a sixth step, Intermediate 3 was treated with tris(dibenzylideneacetone)dipalladium(0), XPhos, bis(pinacolato)diboron and potassium acetate, followed by 1,4-dioxane; Intermediate 16 was then added with aqueous tribasic potassium phosphate solution followed by additional tris(dibenzylideneacctone)dipalladium(0), XPhos and aqueous tribasic potassium phosphate solution to afford tert-Butyl (1-{5-[(7R,14R)-1-(difluoromethoxy)-6-methyl-5-oxo-5,6,7,14-tetrahydro-7,14-methanobenzimidazo[1,2-b][2,5]benzodiazocin-11-yl]pyrimidin-2-yl}cyclobutyl)-carbamate (Intermediate 17) which was purified by flash chromatography. In a seventh step, Intermediate 17 was treated with HCl in 1,4-dioxane followed by aqueous sodium hydroxide solution to yield the title compound as a white amorphous solid. δ_{H} (400 MHz, DMSO-d₆) 9.05 (s, 2H), 8.32-8.22 (m, 1H), 7.91-7.66 (m, 3H), 7.62 (dd, J 8.5, 1.8 Hz, 1H), 7.53-7.46 (m, 2H), 6.31 (d, J 7.1 Hz, 1H), 5.26 (d, J 7.2 Hz, 1H), 3.52 (dt, J 14.2, 7.3 Hz, 1H), 3.36 (s, 3H), 2.84 (d, J 13.8 Hz, 1H), 2.63 (dtd, J 11.5, 5.6, 2.5 Hz, 2H), 2.38 (s, 2H), 2.16-2.05 (m, 2H), 2.04-1.91 (m, 1H), 1.87-1.73 (m, 1H). LCMS (ES+APCI) [M-NH₂]⁻ 486.0, RT 1.66 minutes (Method 2 of WO 2018/197503). LCMS (ES+) [M+H]⁺ 503.0, RT 1.71 minutes (Method 1 of WO 2018/197503).

### Example 2: Clinical Study of Compound 1 in Patients with Mild to Moderate Psoriasis

### Study Design

A double-blind, randomized, placebo-controlled phase 1 study was carried out in participants with mild to moderate psoriasis. Study participants were randomized to 200 mg twice a day (BID) of Compound 1 or placebo in 2:1 ratio (approximately 24 in the treatment group and 12 in the placebo group). The study comprised an up to 4-week screening period, and a 4-week treatment period with Compound 1 or placebo. An end-of-study visit was carried out 10±3 days after the last investigational medicinal product (IMP) administration.

The primary objective of the study was to assess the safety, tolerability, and clinical efficacy of Compound 1 at 200 mg BID via oral administration. Also evaluated was the clinical effect of Compound 1 as compared to placebo on the percent change in PASI and TLS scores from baseline to week 2 and week 4. The percent change in PASI score from baseline to week 4 was determined. TLS score was also assessed over the same time period. Also assessed was the effect of Compound 1 on biomarkers of psoriasis. Blood samples collected at predetermined time points were processed to quantify serum levels of biomarkers, including interleukin (IL)-17A, IL-17F, and IL-22 by high sensitivity methods. IL-17A, IL-17F and IL-22 are the main cytokines released by Th17 cells. Plasma levels of Compound 1 were measured to assess pharmacokinetics in the study population.

### Planned Study Population

Participants were eligible to be included in the study only if all of the following inclusion criteria applied:
I 01. Male or female participants of age between 18 and 65 years, inclusive:
I 02. Confirmed diagnosis of chronic plaque-type psoriasis, diagnosed at least 6 months prior to screening with mild to moderate severity, defined as PASI≤16.
I 03. Participant must have at least two lesions with TLSS≥4 at both screening and baseline excluding the scalp.
I 04. Participant must be in good health (except for psoriasis) as judged by the Investigator, based on medical history, physical examination, vital signs, ECG, clinical laboratories, and urinalysis.
I 05. Female participants who are only postmenopausal or are sterilized (e.g., post-bilateral surgical oophorectomy not linked to a history of cancer) can be included in the study. A postmenopausal state is defined as no menses for 12 months without an alternative medical cause. A high FSH level in the postmenopausal range is used to confirm a postmenopausal state in women (according to the local laboratory) not using hormonal contraception or HRT.
I 06. Laboratory parameters within the normal range at screening (or defined screening threshold for the Investigator site), unless the Investigator considers an abnormality to be clinically irrelevant for the participants; however, hepatic enzymes (ALT, AST), GGT, must not exceed the upper limit of the normal range. Total bilirubin value up to 1.5 the upper normal limit can be acceptable if associated with normal conjugated bilirubin value.
I 07. Normal vital signs after at least 10 minutes resting in supine position at screening:
   90 mmHg≤systolic blood pressure (SBP)≤140 mmHg.
   mmHg≤diastolic blood pressure (DBP)≤90 mmHg.
   50 beats per minute (bpm)≤heart rate (HR)≤100 bpm.
I 08. Standard 12-lead ECG parameters after 10 minutes resting in supine position in the following ranges at screening; PR<220 ms, QRS<120 ms, QTcF≤450 ms (Fridericia QT correction formula, re-checking is not allowed for screening and inclusion), 50 bpm<HR<100 bpm and normal ECG tracing unless the Investigator considers an ECG tracing abnormality to be not clinically relevant.
I 09. Participants of BMI between 18.0 and 35.0 kg/m², inclusive (body weight not under 50.0 kg).
I 10. Male participants should use contraception consistent with local regulations regarding the methods of contraception for those participating in clinical studies. Male participants agree not to donate sperm from the inclusion up to 2.5 months after the last dosing.

Participants were excluded from the study if any of the following criteria applied:
E 01. Pre-existing signs of skin atrophy, telangiectasia or striae in the affected area.
E 02. Current evidence of non-plaque forms of psoriasis (e.g., erythrodermic, guttate or pustular), psoriatic arthritis.
E 03. Currently evidence or suspicion of drug-induced psoriasis (e.g., new onset or exacerbation of psoriasis from beta blockers, calcium channel blockers, or lithium).
E 04. Presence or history of drug hypersensitivity, or allergic disease diagnosed and treated by a physician.
E 05. Opportunistic infections within 6 months before randomization (Day 1).
E 06. Any previous gastrointestinal surgery or recent (within 3 months prior to inclusion Day 1) history of gastrointestinal disease that could impact the absorption of the study drug.
E 07. History of drug or alcohol abuse (more than 800 mL of usual beer or equivalent quantity of approximately 40 g alcohol per day) within the 12 months prior to dosing.
E 08. Evidence of any clinically significant, severe or unstable, acute or chronically progressive, uncontrolled infection or medical condition (including an ongoing biological proven SARS-CoV-2 infection and recurrent infection) or any condition that may affect participant safety in the judgment of the Investigator including participants who are not adequately vaccinated against a SARS-CoV-2 infection according to local regulations.
E 09. Blood donation (any volume) within 2 months before inclusion.
E 10. Known allergy to local anesthetics.
E 11. Use of systemic immunosuppressants within 4 weeks of entering the study and during the entire study duration.
E 12. Use of any of the following therapies within 4 weeks prior to baseline (Day 1): systemic non-biologic psoriasis therapies (including, but not limited to): psoralens and ultraviolet A (PUVA) therapy, cyclosporine, methotrexate, azathioprine, corticosteroids, apremilast, tofacitinib, oral retinoids, mycophenolate mofetil, sirolimus; or phototherapy (including ultraviolet B [UVB] or self-treatment with tanning beds or therapeutic sunbathing) or topical psoriasis therapy with psoralens.
E 13. Use of topical corticosteroid preparations (except hydrocortisone 1%), topical calcineurin inhibitors, or other topical preparations with immunomodulatory properties within 2 weeks prior to randomization (Day 1).
E 14. Prior use of any biologicals for treatment of psoriasis.
E 15. Participants who received any live vaccination within 3 months, any initial non-live vaccination within 30 days or non-live booster vaccination within 14 days of first IMP administration or intend to receive any vaccination during the study.
E 16. Use of any anticoagulants within 3 months prior Day 1 (acetylsalicylic acid within 2 weeks prior Day 1) or during the study.
E 17. Any participant receiving sensitive P-gp, CYP3A4, CYP1A1 and CYP1A2 substrates, respectively, with a narrow therapeutic window or high-risk indication.
E 18. Any participant receiving strong or moderate inducers or inhibitors of CYP3A4 and P-gp within 30 days or 5 half-lives from screening, whichever is longer, prior to enrolment. This also includes the consumption of grapefruit, grapefruit juice, or grapefruit containing products within 72 hours of starting Compound 1 administration.
E 19. Any participant receiving strong or moderate inducers of CYP1A1/CYP1A2 within 30 days or 5 half-lives from screening, whichever is longer, prior to enrolment. This also includes excessive consumption of beverages containing xanthine bases (more than 4 cups or glasses per day).
E 20. Any participant enrolled or having participated in [this or]any other clinical study involving an IMP or in any other type of medical research and is still in the exclusion period according to applicable regulations.
E 21. History of tuberculosis and/or a positive QuantiFERON-TB Gold test (QFT) at Screening.
E 22. Positive for hepatitis B surface antigen and/or Hepatitis B core antibody or positive for anti-human immunodeficiency virus 1 and 2 antibodies (anti-HIV1 and anti HIV2 Ab).
E 23. Hepatitis C antibody positive, unless the participant was successful treated or if the participant was never treated then hepatitis C virus (HCV) RNA by polymerase chain reaction (PCR) should be negative (in both cases, eligibility to be considered with supportive documentation.
E 24. Positive result on urine drug screen (amphetamines/methamphetamines, barbiturates, benzodiazepines, cannabinoids, cocaine, opiates) at the screening.
E 25. Procalcitonin (PCT) higher than 0.5 ng/ml (equivalent to higher than 0.5 ug/L) with signs and symptoms of clinical evident infection at the discretion of the investigator at screening.
E 26. Positive alcohol test at the screening.

Participants were told to avoid direct exposure to natural or artificial sunlight during study treatment and wear protective clothing, sunscreen, and lip balm with a high sun protection factor (e.g., SPF≥30) when outdoors throughout the study duration.

Concerning meals and dietary restrictions, and consumption of caffeine, alcohol and tobacco, the following was stipulated: no excessive consumption (more than 4 cups or glasses per day) of beverages containing methylxanthine bases (e.g., coffee, tea, cola, chocolate, energy drinks) during study participation; no consumption of grapefruits, grapefruit juice, or grapefruit containing products within 72 hours (3 days) before inclusion until end of study participation; no consumption of food or drinks containing poppy seeds within 48 hours prior to screening; smoker and non-smoker participants (according to the randomization list) can be included in the study, restriction is only for smokers with more than 20 cigarettes per day; and no excessive alcohol consumption (i.e., not more than 800 mL of usual beer or equivalent quantity of approximately 40 g alcohol per day) from screening until the end of study participation.

### Study Interventions

Two study interventions were used, Compound 1 and placebo. The Compound 1 intervention comprised 4× tablets of Compound 1 50 mg twice daily, i.e. 200 mg BID, for 4 weeks. The placebo intervention was 4× tablets of placebo twice daily for 4 weeks. Tablets were administered orally.

The interventions were used in two arms of the study. Arm 1 was the experimental arm and it involved participants receiving 200 mg of Compound BID for 4 weeks under fed conditions. Arm 2 was the placebo arm and it involved participants receiving the matching placebo tablets for 4 weeks under fed conditions.

### Study Assessments and Procedures

Planned timepoints for all efficacy assessments were as shown in the Schedule of activities table below:

| | | D1 | | | | | End-of- |
|---|---|---|---|---|---|---|---|
| | | baseline | Study observation period | | | | study |
| Phase | Screening | W 0 | W 1 | W 2 | W 3 | W 4 | W 5 /W 6 |
| VISIT Day ^{a} | D-28 to D-2 | D 1 | D 8 ± 2 | D 15 ± 2 | D 22 ± 2 | D 29 ± 1 | D 38 ± 3 |
| Visits | V01 | V02 | V03 | V04 | V05 | V06 | V07 |
| Time (Hour/minute) ^{b} | | 0 H | 0 H | 0 H | 0 H | 0 H | |
| Informed consent | X | | | | | | |
| Visit at clinical site | X^{c} | X | X | X | X | X | X |
| Overnight stay ^{o} | | X | | | | | |
| Inclusion / exclusion | X | X^{f} | | | | | |
| criteria | | | | | | | |
| Participant | X | | | | | | |
| demography | | | | | | | |
| Medical history ^{r} | X^{d} | X | | | | | |
| Prior / concomitant | < --- - - - - --- > | | | | | | |
| medications | | | | | | | |
| Randomization | | X^{f} | | | | | |
| Diary ^{e} | | X^{f} | X | X | X | X | |
| Compliance check | | | X | X | X | X | |
| (IMP) | | | | | | | |
| Standard breakfast ^{g} | | X^{g} | X^{g} | X^{g} | X^{g} | X^{g} | |
| Study treatment | | | | | | | |
| Compound 1 or | | X----------------------------------------------------------X | | | | | |
| placebo daily oral | | | | | | | |
| tablet administration | | | | | | | |
| (D 1-D 28) | | | | | | | |
| Compound 1 or | | X | | X | | | |
| placebo tablet bottle | | | | | | | |
| dispensation | | | | | | | |
| Return of used or | | | X | X | X | X | |
| unused tablet/ bottles | | | | | | | |
| Safety ^{k} | | | | | | | |
| Physical examination | X | X^{f} | X | X | X | X | X |
| Height | X | | | | | | |
| Body weight | X | X^{f} | | X | | X | X |
| Auricular body | X | X^{f} | X^{f} | X^{f} | X^{f} | X | X |
| temperature | | | | | | | |
| Vital signs | X | X^{f} | X^{f} | X^{f} | X^{f} | X | X |
| 12-lead ECG ^{q} | X | X^{f p} | X^{f} | X^{f p} | X^{f} | X^{f p} | X |
| Archival blood | | X^{f} | | | | | |
| sample | | | | | | | |
| Hematology, | X | X^{f} | X^{f} | X^{f} | X^{f} | X | X |
| biochemistry, | | | | | | | |
| coagulation, | | | | | | | |
| urinalysis | | | | | | | |
| Serology tests | X | | | | | | |
| Urine drug screen, | X | | | | | | |
| alcohol breath test | | | | | | | |
| COVID-19 test ^{h} | X | X^{s} | | | | | |
| QuantiFERON-TB | X^{c} | | | | | | |
| Gold test | | | | | | | |
| Procalcitonin | X | | | X | | | X |
| β-HCG blood test (if | X | X | | | | X | |
| applicable) | | | | | | | |
| Urinary pregnancy | X | X^{f} | | | | X | |
| test | | | | | | | |
| FSH (if applicable) | X | | | | | | |
| Adverse event | <- - - - - ---> | | | | | | |
| collection | | | | | | | |
| Clinical response ^{l} | | | | | | | |
| Target lesion severity | X | X^{f} | | X^{f} | | X | |
| score (TLSS) | | | | | | | |
| Psoriasis Area and | X | X^{f} | | X^{f} | | X | |
| Severity Index (PASI) | | | | | | | |
| (Static) Investigator's | X | X^{f} | | X^{f} | | X | |
| Global Assessment | | | | | | | |
| (sIGA) | | | | | | | |
| Photographs of | | X^{f} | | | | X | |
| psoriasis plaque ^{j} | | | | | | | |
| Pharmacokinetics | | | | | | | |
| Compound 1 blood | | P01 | P03^{f} | P04^{f} + P05 | P06^{f} | P07^{f} + P08 | |
| samples | | post dose | | post dose | | post dose | |
| | | (3 h ±1 h) + | | (3 h ± 1 h) | | (3 h ± 1 h) | |
| | | P02ⁿ | | | | | |
| 4β- | | X^{f} | | X^{f} | | X^{f} | |
| hydroxycholesterol | | | | | | | |
| blood samples | | | | | | | |
| DNA (optional) | | | | | | | |
| DNA | | X^{f} | | | | | |
| pharmacogenetic | | | | | | | |
| sample for banking | | | | | | | |
| Pharmacodynamics | | | | | | | |
| Pharmacodynamic | | X^{f} | | X^{f} | | X^{m} | |
| blood samples for | | | | | | | |
| hsCRP (included in | | | | | | | |
| the safety laboratory) | | | | | | | |
| Pharmacodynamic | | X^{f} | | X^{f} | | X^{m} | |
| blood samples for | | | | | | | |
| IL-17A | | | | | | | |
| Pharmacodynamic | | X^{f} | | X^{f} | | X^{m} | |
| blood samples for | | | | | | | |
| IL-17F | | | | | | | |
| Pharmacodynamic | | X^{f} | | X^{f} | | X^{m} | |
| blood samples for | | | | | | | |
| IL-22 | | | | | | | |
| Skin biopsy (optional) ⁱ | | X^{f} | | | | X^{m} | |

Abbreviations: D = Day, h = hours, V = Visit, W = week
^{a} If the participant discontinues early from treatment, the participant is to be assessed using the safety and clinical response procedure planned for the end-of-treatment study visit at week 4, including the blood samples collection for pharmacokinetics and pharmacodynamics assessment if appropriate
^{b} Time (hour/minute) is expressed in reference to the last administration of Compound 1 (T0 H)
^{c}Visit to be scheduled appropriately since laboratory results (in particular QuantiFERON-TB Gold test) at screening will be released after a minimal time limit of 1 week after blood sample reception at the Lab
^{d}Including vaccination history and tonsillitis/perionditis occurring within 4 weeks
^{e} Diary provided to the trial participant on D 1 and at each visit, then returned and checked by the investigator to verify the IMP intake and validate the adverse events reported on Day 8 ± 2, Day 15 ± 2, Day 22 ± 2, and Day 29 ± 1
^{f}Pre-dose
^{g}Dosing on Day 1, Day 8, Day 15, and Day 22 will be on study site, a standard hospital breakfast (moderate-fat meal) will be served by study site before study drug dosing i.e., study drug administration approx. 5 to 15 min after the end of the breakfast, however the study medication can be taken at home just before a meal or during a meal or immediately after a meal. Study drug should be administered with a glass of water (min. 240 ml).
^{h} Covid-19 test might be repeated during the study as needed according to the investigator's opinion and according to national and local regulations if applicable, the test at screening as nasopharyngeal swab will be analyzed by PCR, the PCR test can be also performed outside the study site, New covid-19 test may be used during the study according to available scientific knowledge and or local health authority recommendation.
ⁱ Skin biomarkers (RNA analysis) from biopsies (3 mm punch): prior to first dosing (Day 1) in lesional and non-lesional parts, and at week 4 only in lesional locations, biopsy is an optional for participants who agreed and have visible psoriatic plaque in a region suitable for tissue biopsy
^{j} Photography is optional, more details are provided in the Photography Reference Manual
^{k} Refer to Safety section (Section 8.2) for detailed safety investigations
^{l} TLSS, PASI, sIGA should always be completed by the same physician for a given participant, if possible, in order to secure longitudinal comparison and avoid any bias
^{m}Pre-dose at Day 28 (in case the site visit on Day 28) if the site visit on Day 29 or Day 30 it should be at the same dosing time of the treatment period.
ⁿPre-dose of evening IMP administration (8-12 hours after morning IMP dose)
^{o} Overnight stay is optional from Day −1 to Day 1 and/or Day 1 to Day 2 for participants who are living far from the study site
^{p} ECG at post dose (3 h ± 1 h)
^{q} Triplicate digital ECGs to be recorded within 5 minutes with at least 1 min between 2 replicates for all ECGs.
^{r} Medical history includes but are not limited to surgical history/Psoriasis history/Tonsillitis/periodontitis history/Smoking Status/concomitant disease(s)
^{s}Covid-19 test may be done on Day −1 in case of overnight stay from Day −1 to Day 1
Note:
when several items take place at the same time, the following order should be respected: ECG, vital signs, questionnaires, pharmacodynamics, blood sampling, meal (if applicable), drug administration; the exact timing of pharmacokinetic samples, should be respected.

TLS score, which is a validated tool for psoriasis severity assessment, was used to evaluate severity and to assess clinical efficacy. It consists of a sum score of 3 clinical severity parameters (scaling, erythema, plaque elevation/induration; each graded on a 5-point severity scale; 0=clear, 1=slight, 2=mild, 3=moderate, and 4=severe) with a sum score ranging between 0 and 12 points (see, e.g., Czamowicki et al., J Am Acad Dermatol. (2014) 71(5):954-959.e1). The following assessment was used, in which TLS score was calculated as the sum of A+B+C (adapted from Fredriksson et al., Dermatologica (1978) 157(4):238-244):

| A- Scaling | B- Erythema | C- Plaque Elevation |
|---|---|---|
| 0 - Clear: no scale | 0 - No erythema: | 0 - No plaque elevation: no elevation |
| | hyperpigmentation, diffused faint | above normal skin |
| | pink or red coloration | |
| 1 - Slight scaling: surface dryness | 1 - Slight: up to moderate, up to | 1 - Slight: difficult to ascertain |
| with some white coloration | definite red coloration | whether there is a slight |
| | | elevation above normal skin |
| 2 - Mild scaling: fine scales | 2 - Mild: up to moderate, up to | 2 - Mild: light but definite elevation; |
| partially or mostly covering lesions | definite red coloration | typically edges are indistinct or sloped |
| 3 - Moderate scaling: coarse scales, | 3 - Moderate: moderate, definite | 3 - Moderate: moderate elevation with |
| covering most or all the lesions | red coloration | rough or sloped edges |
| 4 - Severe scaling: coarse; | 4 - Severe: severe, very bright | 4 - Severe: marked elevation typically |
| tenacious scale predominates | red coloration | with hard or sharp edges |
| covering most or all lesions | | |

The PASI scoring system evaluates psoriasis based on the body surface area (BSA) involvement and morphological characteristics of the plaques (see, e.g., EMA Guidelines on clinical investigation of medicinal products indicated for the treatment of psoriasis, Nov. 18, 2004). BSA estimation was performed using the ‘palm’ (i.e., the subject's flat hand and thumb together, fingers not included). The palm thus defined represented 1% of the total BSA. The PASI combines assessments of four body areas: head (h, 10%), upper extremities (u, 20%), trunk (t, 30%) and lower extremities (1, 40%). Using the palm to assess BSA (as described above) the proportion of skin affected by psoriasis in each area was given a numerical score (A) representing the proportion involved: 1 (0 to 9%); 2 (10 to 29%); 3 (30 to 49%); 4 (50 to 69%); 5 (70 to 89%) and 6 (90 to 100%). Within each area the severity of three signs, erythema (E), thickness/induration (I), and desquamation/scaling (D), was each assessed on a five-point scale: 0, none; 1, mild; 2, moderate; 3, severe; 4, very severe. The PASI ranges from 0 to 72 and is calculated using the following formula:

$$PASI = 0.1 ( Eh + Ih + Dh ) Ah + 0.2 ( Eu + Iu + Du ) Au + 0.3 ( Et + It + Dt ) At + 0.4 ( El + Il + Dl ) Al$$

The sIGA employed in this study is a six-point ordinal scale used to assess the global severity of disease over the body as a whole, as measured by the overall degree of induration, scaling and erythema of a subject's psoriatic lesions. For statistical analysis, the scale was assigned scores of 0 to 5, for ‘clear’ to ‘very severe’, respectively.

Scoring was accorded to a subject on a scale of 0 to 5 for each of erythema, induration, and scaling; a mean sIGA score was calculated as the mean average of those individual scores and this was translated into an overall sIGA score (an integer between 0 and 5) as described in the tables below:

| | | |
|---|---|---|
| Erythema | 0 | Normal (post-inflammatory hyperpigmentation may be |
| | | present) |
| | 1 | Normal to pink coloration |
| | 2 | Pink to light red coloration |
| | 3 | Dull to bright red, clearly distinguishable erythema |
| | 4 | Bright to deep dark red coloration |
| | 5 | Deep dark red coloration |
| Induration | 0 | None |
| | 1 | No thickening |
| | 2 | just detectable mild thickening |
| | 3 | Clearly distinguishable to moderate thickening |
| | 4 | Severe (marked) thickening with hard edges |
| | 5 | Very severe thickening with hard edges |
| Scaling | 0 | No scales |
| | 1 | No to minimal focal scaling |
| | 2 | Predominantly fine scaling |
| | 3 | Moderate (mild to coarse) scaling |
| | 4 | Severe (coarse) scaling covering almost all lesions |
| | 5 | Very severe (very coarse) scaling covering all lesions |

| Overall | | |
|---|---|---|
| Score | Calculation | Detailed description |
| 0 | 0 for all three | No signs of psoriasis |
| (Clear) | | Post-inflammatory hyperpigmentation may be |
| | | present |
| 1 | 0 < mean > 1.5 | No thickening |
| (Almost clear / | | Normal pink coloration |
| Minimal) | | No to minimal focal scaling |
| 2 | 1.5 < mean > 2.5 | Just detectable mild thickening |
| (Mild) | | Pink to light red coloration |
| | | Predominantly fine scaling |
| 3 | 2.5 < mean > 3.5 | Clearly distinguishable to moderate thickening |
| (Moderate) | | Dull to bright red, clearly distinguishable erythema |
| | | Moderate (mild to coarse) scaling |
| 4 | 3.5 < mean > 4.5 | Severe (marked) thickening with hard edges |
| (Severe) | | Bright to deep dark red coloration |
| | | Severe (coarse) scaling covering almost all lesions |
| 5 | 4.5 < mean | Very severe thickening with hard edges |
| (Very severe) | | Deep dark red coloration |
| | | Very severe (very coarse) scaling covering all |
| | | lesions |

Planned timepoints for all safety assessments are shown in the schedule of activities table above.

Physical examination included at a minimum: heart and respiratory auscultation; peripheral arterial pulse; pupil, knee, Achilles, and plantar reflexes; peripheral lymph nodes and abdomen examination.

Vital signs (HR, SBP and DBP measured after at least 10 minutes in supine resting position and after approximately 3 minutes in standing position), and auricular body temperature were measured.

The following electrocardiogram data were recorded:
Standard 12-lead ECGs (safety ECGs) were recorded after at least 10 minutes in supine position using an electrocardiographic device. The electrodes were positioned at the same place for each ECG recording throughout the study (e.g., attachment sites of the leads were visualized on a transparent paper).
Triplicate ECGs were recorded within 5 minutes with at least 1 min between 2 replicates for all ECG.
Each ECG consisted of a 10-second recording of the 12 leads simultaneously, leading to:
A single 12-lead ECG (50 mm/s, 10 mm/mV) printout with heart rate, PR, QRS, QT, QTc automatic correction evaluation (by the ECG device), including date, time, and number of the participant, signature of the research physician, and at least 3 complexes for each lead. The Investigator's medical opinion and automatic values were recorded in the eCRF. This printout was retained at the site. A second printout was performed also, if needed.
A digital storage that enables eventual further reading by an ECG central laboratory: each digital file will be identified by theoretical time (day and time Dxx Txx Hxx), real date and real time (recorder time), Sponsor study code, participant number (i.e., 3 digits), and site and country numbers, if relevant. The digital recording, data storage, and transmission (whenever requested) need to comply with all applicable regulatory requirements (i.e., FDA 21 code of federal regulations [CFR], Part 11).

Laboratory tests (from at least 10 hours under fasting conditions for blood samples) included:
Hematology: red blood cell count, hematocrit, hemoglobin, white blood cell count with differential count (neutrophils, eosinophils, basophils, monocytes, and lymphocytes), platelets.
Biochemistry:
Plasma/serum electrolytes: sodium, potassium, chloride, calcium,
Liver function: AST, ALT, alkaline phosphatase, GGT, total and conjugated bilirubin,
Renal function: urea, creatinine,
Metabolism: glucose, albumin, total proteins, total cholesterol, triglycerides,
Potential muscle toxicity: CK,
International normalized ratio (INR), and activated partial thromboplastin time (aPTT),
C-reactive protein; the CRP levels were evaluated by an independent investigator who is not involved in the study to protect the blinding of the study. CRP values were provided by the independent investigator to the investigator in case of medical need.
Procalcitonin at screening, week 2 and EOS.
QuantiFERON-TB Gold (QFT) test.

If the participant was female, serum β-HCG test was required at screening, Day 1 and Day 29±1 and urine pregnancy test is performed at screening, Day 1 and Day 29±1. If postmenopausal female, plasma or serum FSH was tested at screening only.

Urinalysis included specific gravity, proteins, glucose, blood, bilirubin, urobilinogen, leucocytes, ketone bodies, nitrite and pH:
If any parameter on the dipstick was abnormal, quantitative measurement for urine was performed. If positive for protein and/or red blood cells, microscopic analysis was performed by the local laboratory.

Urine drug screening was done for amphetamines/methamphetamines, barbiturates, benzodiazepines, cannabinoids, cocaine, and opiates (only at screening).

Serology tests included hepatitis B antigen, anti-hepatitis B core antibodies (anti-HBc Ab), hepatitis C antibodies, anti-HIV1 and anti-HIV2 antibodies.

A 15 mL (or three 5 mL) blood sample(s) were collected into a dry, red topped tube, kept upright at room temperature for 30 minutes and then centrifuged at approximately 1500 g for 10 minutes at 4° C.; the serum was then transferred into 3 storage tubes, which were immediately capped and frozen in an upright position at 20° C. This sample was to be used if any unexpected safety issue occurred to ensure that a pre-administration baseline value is available for previously non assessed parameters (e.g., serology). If this sample was not used, the Investigator would destroy it after the Sponsor's approval.

A COVID-19 test could be repeated during the study as needed according to the Investigator's opinion and according to national and local regulations if applicable. The test at screening was a nasopharyngeal swab that was analyzed by PCR.

Other assessments included body weight (kg), auricular body temperature, and alcohol breath test (only at screening).

Note: When vital signs, ECG and blood samples and skin biopsies at Day 1, are scheduled at the same time as the IMP administration and/or a meal, they are done prior to IMP intake and/or meal. In addition, blood PK sampling at the scheduled visits (Day 1, Day 15+2 and Day 29±1) at pre-dose as well as the 3-hour post dosing. All other safety, pharmacodynamics and clinical response assessments scheduled at weeks 2 and 4 are performed close to dosing (before or after).

Adverse events (AEs) were reported by the participant or observed by the Investigator, and were defined as follows:
An AE is any untoward medical occurrence in a patient or clinical study participant, temporally associated with the use of study intervention, whether or not considered related to the study intervention.
NOTE: An AE can therefore be any unfavorable and unintended sign (including an abnormal laboratory finding), symptom, or disease (new or exacerbated) temporally associated with the use of study intervention.

Events meeting the AE definition:
Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the Investigator (i.e., not related to progression of underlying disease), e.g.:
Symptomatic and/or
Requiring either corrective treatment or consultation, and/or
Leading to IMP discontinuation or modification of dosing, and/or
Fulfilling a seriousness criterion, and/or
Defined as an AESI
Exacerbation of a chronic or intermittent pre-existing condition including either an increase in frequency and/or intensity of the condition.
New condition detected or diagnosed after study intervention administration even though it may have been present before the start of the study.
Signs, symptoms, or the clinical sequelae of a suspected drug-drug interaction.
Signs, symptoms, or the clinical sequelae of a suspected overdose of either study intervention or a concomitant medication.
Lack of efficacy or failure of expected pharmacological action per se will not be reported as an AE or SAE. Such instances will be captured in the efficacy assessments. However, the signs, symptoms, and/or clinical sequelae resulting from lack of efficacy will be reported as AE or SAE if they fulfill the definition of an AE or SAE.

Events NOT meeting the AE definition:
Any clinically significant abnormal laboratory findings or other abnormal safety assessments that are associated with the underlying disease, unless judged by the Investigator to be more severe than expected for the participant's condition.
The disease/disorder being studied or expected progression, signs, or symptoms of the disease/disorder being studied, unless more severe than expected for the participant's condition.
Medical or surgical procedure (e.g., endoscopy, appendectomy): the condition that leads to the procedure is the AE.
Situations in which an untoward medical occurrence did not occur (social and/or convenience admission to a hospital).
Anticipated day-to-day fluctuations of pre-existing disease(s) or condition(s) present or detected at the start of the study that do not worsen.

A serious adverse event (SAE) is any untoward medical occurrence that at any dose, meets one or more of the criteria listed:
A) Results in death
B) Is life-threatening
   Note: The term “life-threatening” in the definition of “serious” refers to an event in which the participant was at risk of death at the time of the event; it does not refer to an event which hypothetically might have caused death if it were more severe.
C) Requires in participant hospitalization or prolongation of existing hospitalization In general, hospitalization signifies that the participant has been admitted (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether hospitalization occurred or was necessary, the AE should be considered serious.
   Hospitalization for elective treatment of a pre-existing condition that did not worsen from baseline is not considered an AE.
D) Results in persistent or significant disability/incapacity
   The term disability means a substantial disruption of a person's ability to conduct normal life functions.
   This definition is not intended to include experiences of relatively minor medical significance such as uncomplicated headache, nausea, vomiting, diarrhea, influenza, and accidental trauma (e.g., sprained ankle) that may interfere with or prevent everyday life functions but do not constitute a substantial disruption.
E) Is a congenital anomaly/birth defect
F) Is a medically important event:
   Medical and scientific judgment should be exercised in deciding whether expedited reporting is appropriate in other situations, such as important medical events that may not be immediately life-threatening or result in death or hospitalization but may jeopardize the participant or may require medical or surgical intervention to prevent one of the other outcomes listed in the definition above.
   Note: Examples of such events are intensive treatment in an emergency room or at home for allergic bronchospasm, blood dyscrasias, convulsions, ALT>3×ULN+total bilirubin>2×ULN or asymptomatic ALT increase >10×ULN, development of drug dependence or drug abuse, suicide attempt or any event suggestive of suicidality, syncope, loss of consciousness (except if documented as a consequence of blood sampling) or bullous cutaneous eruptions.

The Investigator would assess the intensity for each AE and SAE reported during the study and assign it to 1 of the following categories (as per NCI-CTCAE):
Grade 1: Mild; asymptomatic or mild symptoms; clinical or diagnostic observations only; intervention not indicated.
Grade 2: Moderate; minimal, local, or noninvasive intervention indicated; limiting age-appropriate instrumental activities of daily living (ADL). Instrumental ADL refer to preparing meals, shopping for groceries or clothes, using the telephone, managing money, etc.
Grade 3: Severe or medically significant but not immediately life-threatening; hospitalization or prolongation of hospitalization indicated; disabling; limiting self-care ADL. Self-care ADL refer to bathing, dressing, and undressing, feeding self, using the toilet, taking medications and not bedridden.
Grade 4: Life-threatening consequences; urgent intervention indicated.
Grade 5: Death related to AE.

An event was defined as “serious” when it met at least 1 of the predefined outcomes as described in the definition of an SAE, not when it was rated as severe.

Whenever possible, diagnosis or single syndrome was reported instead of symptoms. The Investigator would specify the date of onset, intensity, action taken with respect to IMP, corrective treatment/therapy given, additional investigations performed (e.g., in the case of dermatologic lesions photographs were required), outcome, and Investigator's opinion as to whether there is a reasonable possibility that the AE was caused by the IMP.

All AEs (serious or nonserious) were collected from the signing of the informed consent form (ICF) until EOS at the timepoints specified in the Schedule of activities above. All SAEs and AESI were recorded and reported to the Sponsor or designee immediately and under no circumstance after 24 hours. The Investigator submitted any updated SAE data to the Sponsor within 24 hours of it being available. Investigators were not obligated to actively seek information on AEs or SAEs after conclusion of the study participation. However, if the Investigator learned of any SAE, including a death, at any time after a participant had been discharged from the study, and he/she considered the event to be reasonably related to the study intervention or study participation, the Investigator was required to notify the Sponsor promptly.

Care was taken not to introduce bias when detecting AEs and/or SAEs. Open-ended and nonleading verbal questioning of the participant was the preferred method to inquire about AE occurrences.

All AEs, regardless of seriousness or relationship to IMP/non-investigational medicinal product (NIMP), spanning from the signature of the informed consent form until the end of the study as defined by the protocol, were recorded on the corresponding page(s) or screen(s) of the case report form for included participants. For screen failed participants, recording in the case report form was only performed in case of SAE occurring during the screening period or in case of AE when some screening procedures exposed the participant to safety risks (e.g., any substance administered as pretreatment or for phenotyping, invasive tests performed, or chronic treatment interrupted).

In order to ensure the safety of the participants, the Investigator took appropriate measures to follow all AEs until clinical recovery was complete and laboratory results returned to normal, or until progression stabilized, or until death. This may imply that observations would continue beyond the last planned visit per protocol, and that additional investigations may be requested by the monitoring team.

When treatment was prematurely discontinued, participants were assessed using the procedure planned for the end-of-study visit, including a pharmacokinetic sample if appropriate as defined by the protocol. Laboratory, vital signs, or ECG abnormalities were recorded as AEs in the eCRF if.
symptomatic, and/or
requiring either corrective treatment or consultation, and/or
leading to IMP/NIMP discontinuation or modification of dosing, and/or fulfilling a seriousness criterion, and/or defined as an AESI.

Pregnancy occurring in a female partner of a male participant included in the study was qualified as a SAE only if meeting one of the seriousness criteria. In the event of pregnancy of a female participant, IMP was discontinued. If pregnancy occurs during screening phase, it was reported only if it met one of the seriousness criteria. Follow-up of pregnancy was mandatory until its outcome had been determined. If a pregnancy was reported, the Investigator recorded pregnancy information on the appropriate form and submitted it to the Sponsor within 24 hours of learning of the female participant.

Cardiovascular events were reported if they met AE/SAE criteria. Events with fatal outcome events were reported as SAEs.

An adverse event of special interest (AESI) is an AE (serious or non-serious) of scientific and medical concern, specific to the IMP or program, for which ongoing monitoring and rapid communication by the Investigator to the Sponsor may be appropriate. Such events may require further investigation in order to characterize and understand them. The AESI may be added, modified or removed during a study by protocol amendment.

For AESI in this study, the Sponsor was to be informed immediately (i.e., within 24 hours), as per SAE notification guidelines, even if a seriousness criterion was not met, using the corresponding screens in the eCRF.
ALT increased >3×ULN.
QT prolongation (QTcF≥500 milli seconds [ms]) and/or a delta QTc change from baseline>60 ms (12-lead ECG). In the event of prolongation of QTc interval (automatic measurement)≥500 ms, confirmed by a manual reading by the Investigator or a physician delegated by the Investigator using the Fridericia formula for correcting QT, the participant was placed under supervision in a specialized setting. Investigational medicinal product administration was stopped, and appropriate blood samples collected. Subsequent ECG monitoring of the participant was then performed on a regular and clinically responsible basis until the QTc interval returned to a safe value as determined by the Investigator in agreement with the Sponsor.
Photosensitivity (Grade 2 and above AE due to sun exposure).
Pregnancy.
Symptomatic overdose.
   An overdose (accidental or intentional) with the IMP is an event suspected by the Investigator or spontaneously notified by the participant (not based on systematic pills count) and defined as at least twice the intended dose within the intended therapeutic interval, adjusted according to the tested drug.

Of note: Asymptomatic overdose had to be reported as standard AE.

Once the participant was included in the clinical trial, the following laboratory abnormalities were monitored, documented, and managed:
Neutropenia, characterized as neutrophils <1500/mm³ or according to ethnic group (recorded as an AE only if at least 1 of the criteria listed above was met)
Thrombocytopenia, characterized as platelets <100,000/mm³ confirmed with or without bleeding (recorded as an AE only if at least 1 of the criteria listed above was met)
Increase of ALT. Patients were monitored where 3 ULN<ALT≤5 ULN and total bilirubin was ≤2 ULN. IMP was discontinued where total bilirubin was >2 ULN or ALT>5 ULN.
Acute renal impairment, characterized as persistent increase from baseline in creatine of 0.3 mg/dL (~26.4 pmol/L) or 30%, whichever is less (recorded as an AE only if at least 1 of the criteria listed above was met)
Increase in CPK suspected to be on non-cardiac origin and not related to an intensive physical activity, identified by muscular symptoms or systematic assessment and characterized as CPK>3 ULN (Suspicion of rhabdomyolysis was recorded as AE only if at least 1 of the criteria listed above was met; increase in CPK was recorded as an AE only if at least 1 of the criteria listed above was met)

Blood samples were processed for analysis of IL-17A, IL-17F and IL-22 in serum samples by high sensitivity methods. Samples were collected according to the schedule of activity described above.

### Results—Patient Demographics and Baseline Characteristics

A total of 38 participants were randomized; 26 were exposed to Compound 1, and 12 to placebo. Thirty six participants completed the study. Two participants having received Compound 1 at 200 mg BID withdrew due to AEs (one on Day 8 and one on Day 15), and were replaced to complete 36 participants for the pharmacodynamic population. Safety data were available for all 38 participants, clinical response data were available for 37 participants (only one of the two dropped out participants had one post-baseline PD assessment on Day 15), and biomarker data were available for 38 participants (the two dropped out participants had post-baseline biomarker assessments on Day 8 and only for one on Day 15).

Thirty eight male participants with comparable demographic characteristics were enrolled (demographics are summarized in Table 1 below). The median age was 43, more particularly 42.5, (range 21-64) with a median BMI of 26.2 kg/m² (range 21.0-32.7). All had confirmed diagnosis (at least 6 months prior to screening) of chronic plaque psoriasis and presented with at least two lesions with TLS score ≥4 at the screening and baseline. Mean (±SD) baseline TLS score was about 7.0, more particularly 7.01, (+1.54) and mean PASI was 8.6, more particularly 8.58, (±3.40) at baseline. A total of 13/38 (34.2%) participants had total PASI score ≥10 (moderate psoriasis). A total of 25/38 (65.8%) participants had total PASI score <(mild psoriasis). Participants were prohibited from using any systemic or topical psoriasis therapies except the use of topical hydrocortisone up to 1% was allowed, treatment with 1,25-dihydroxyvitamin D analogs could be kept at a stable dose until the end of study if started 6 months before Day 1. Nevertheless, none of the participants used allowed treatment during the study.

TABLE 1
Demographics and baseline characteristics of study participants

| Baseline Characteristics | Placebo (n = 12) | Compound 1 (n = 26) |
|---|---|---|
| Age, mean (±SD), years | 40.5 (12.5) | 44.2 (9.7) |
| BMI, mean (±SD), kg/m² | 25.98 (2.92) | 26.45 (2.97) |
| TLS score, mean (±SD) | 7.42 (1.40) | 6.83 (1.60) |
| PASI, mean (±SD) | 7.86 (2.53) | 8.91 (3.73) |
| sIGA score | 2.25 (0.45) | 2.42 (0.64) |
| PASI score, n (%) | | |
| <10 (mild psoriasis) | 8 (66.7) | 17 (65.4) |
| ≥10 (moderate psoriasis) | 4 (33.3) | 9 (34.6) |

### Results—Clinical Response by TLS Score

The TLS score was assessed at baseline, 2 weeks and 4 weeks. Table 2 below presents the Least Square means (LSm)±Standard Error (SE) for each treatment and the Least Squares means difference (ΔLSm; Compound 1 minus placebo)±Standard Error (SE) with 90% confidence interval (CI) in percentage improvement from baseline in the TLS score (calculated as the average of the TLS score values collected from 2 lesions) at week 2 and week 4.

TABLE 2
Adjusted mean % improvement and 90% CI from baseline
in TLS score between Compound 1 and placebo

| Comparison | LSm | LSm | | |
|---|---|---|---|---|
| vs. | (±SE) for | (±SE) for | | |
| baseline | Compound 1 | placebo | ΔLSm (±SE) | 90% CI |
| At week 2 | 17.06 (3.13) | 6.29 (4.68) | 10.77 (5.61) | (1.29 to 20.26) |
| At week 4 | 38.18 (4.33) | 20.44 (6.18) | 17.74 (7.54) | (4.98 to 30.49) |

The results in Table 2 were obtained using the Mixed Model for Repeated Measures (MMRM).

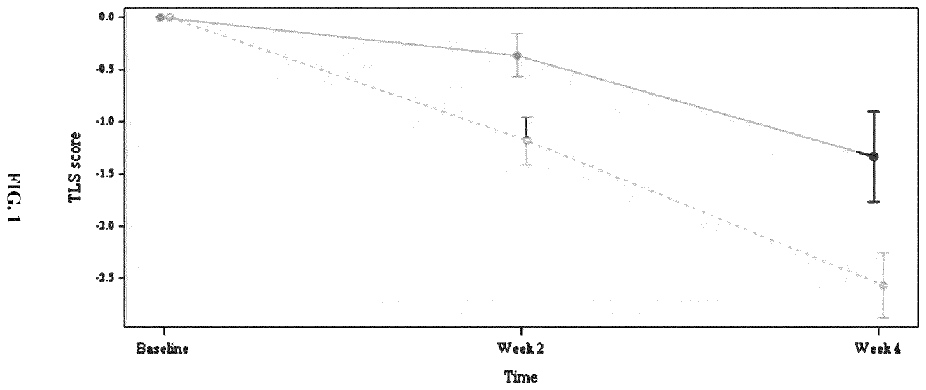

FIG. 1 shows the mean (±SEM) change in TLS score from baseline over time. The placebo treatment arm (solid line with filled circles) shows a mean reduction of around 1.25 points at week 4, whereas the Compound 1 treatment arm (dashed line with open circles) shows a mean reduction of around 2.5 points over the same period.

Table 3 below presents the mean (±SEM) percentage improvement from baseline in the TLS score (calculated as the average of the TLS score values collected from 2 lesions) at week 2 and week 4 in the patient population with PASI<10 (mild) or PASI≥10 (moderate) at baseline.

TABLE 3
Mean (±SEM) % improvement from baseline
in TLS score in subpopulations

| | PASI < 10 (mild) | | | PASI ≥ 10 | | |
|---|---|---|---|---|---|---|
| | population | | | (moderate) population | | |
| | | Compound 1 | | | | Compound 1 |
| TLS | Placebo | 200 mg BID | | Placebo | | 200 mg BID |
| score | (N = 8) | (N = 16) | | (N = 4) | | (N = 9) |
| Week | 8.5 (4.35) | 20.9 | (3.61) | 0.0 | (0.00) ⁽¹⁾ | 10.9 (6.20) |
| 2 | | | | | | |
| Week | 22.3 (9.0) | 37.0 | (6.32) ⁽²⁾ | 15.0 | (7.97) | 39.7 (4.03) |
| 4 | | | | | | |

The results in Table 3 come from standard descriptive analysis, ⁽¹⁾N=3, ⁽²⁾N=15

Based on the observed data, the difference in percentage mean improvement of TLS score at week 4 between Compound 1 and placebo was estimated to be 17.7% (90% CI [5.0%; 30.5%]).

### Results—Clinical Response by PASI Score

The PASI score was assessed at baseline, 2 weeks and 4 weeks. Table 4 below presents the LSm (±SE) for each treatment and ΔLSm (Compound 1 minus placebo) with 90% confidence interval (CI) in percentage improvement from baseline in the total PASI score at week 2 and week 4.

TABLE 4
Adjusted mean % improvement and 90% CI from baseline
in total PASI score between Compound 1 and placebo

| Comparison | LSm | LSm | | |
|---|---|---|---|---|
| vs. | (±SE) for | (±SE) for | | |
| baseline | Compound 1 | placebo | ΔLSm (±SE) | 90% CI |
| At week 2 | 17.73 (2.75) | 4.12 (4.10) | 13.61 (4.92) | (5.28 to 21.94) |
| At week 4 | 35.09 (4.47) | 15.71 (6.33) | 19.38 (7.75) | (6.28 to 32.48) |

The results in Table 4 were obtained using the MMRM.

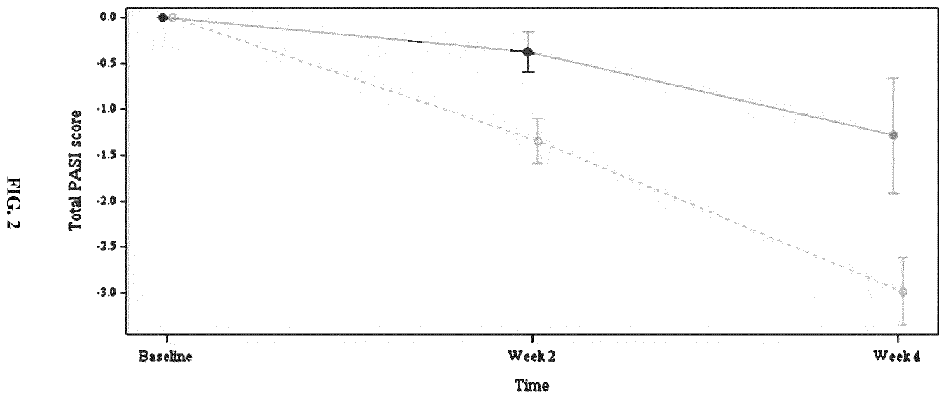

FIG. 2 shows the mean (±SEM) change in total PASI score from baseline over time. The placebo treatment arm (solid line with filled circles) shows a mean reduction of around 1.25 points at week 4, whereas the Compound 1 treatment arm (dashed line with open circles) shows a mean reduction of around 3.0 points over the same period.

Table 5 below presents the mean (±SEM) percentage improvements from baseline in the total PASI score at week 2 and week 4 in the patient population with PASI<10 (mild) or PASI≥(moderate) at baseline.

TABLE 5
Mean (±SEM) % improvement from baseline
in total PASI score in subpopulations

| | PASI < 10 | | | PASI ≥ 10 | | |
|---|---|---|---|---|---|---|
| | (mild) population | | | (moderate) population | | |
| | | Compound 1 | | | | Compound 1 |
| | Placebo | 200 mg BID | | Placebo | | 200 mg BID |
| PASI | (N = 8) | (N = 16) | | (N = 4) | | (N = 9) |
| Week 2 | 4.8 (3.89) | 21.2 | (3.98) | 3.5 | (1.43)⁽¹⁾ | 9.3 (3.60) |
| Week 4 | 14.3 (11.66) | 33.8 | (5.34)⁽²⁾ | 19.9 | (8.83) | 33.2 (4.03) |

The results in Table 5 come from standard descriptive analysis, ⁽¹⁾N=3, ⁽²⁾N=15.

Based on the observed data, the difference in percentage mean improvement of total PASI score at week 4 between Compound 1 and placebo was estimated to be 19.4% (90% CI [6.3%; 32.5%]). The improvement in PASI score after 4 weeks of treatment with Compound 1 is clinically meaningful and is consistent with the improvement in the TLS score in mild-to-moderate psoriasis.

Overall, Compound 1 at 200 mg BID showed a significant improvement of total PASI score at week 2 (17.73% versus 4.12%, p=0.005) and at week 4 (35.09% versus 15.71%, p=0.009) (% mean improvement from baseline, p-value for one sided test at 5% significance level). Consistently, there was a clear improvement in the TLS score from baseline to week 2 (17.06% versus 6.29%, p=0.032) and week 4 (38.18% versus 20.44%, p=0.012). A separate analysis of patients with mild (PASI<10) or moderate (PASI≥10) psoriasis showed improvement at week 2 (mild: 20.9%; moderate: 10.9%) and week 4 (mild: 37.0%; moderate: 39.7%) despite disease severity.

Results—Clinical Response by sIGA Score

The sIGA score was assessed as described herein. As shown in Table 6 below, the proportion of patients achieving improvement by at least 1 severity level in sIGA score measured at week 4, as compared to sIGA measurement at baseline, was 58.3% in the treatment arm vs 0% in the placebo group.

TABLE 6
Number (%) of subjects with change in sIGA score from baseline

| sIGA change from baseline | | −1 | No change | +1 |
|---|---|---|---|---|
| Placebo | Week 2 | 0 | 10 (90.9%) | 1 (9.1%) |
| | Week 4 | 0 | 10 (83.3%) | 2 (16.7%) |
| Compound 1 | Week 2 | 8 (32.0%) | 17 (68.0%) | 0 |
| 200 mg BID | Week 4 | 14 (58.3%) | 9 (38.5%) | 1 (4.2%) |

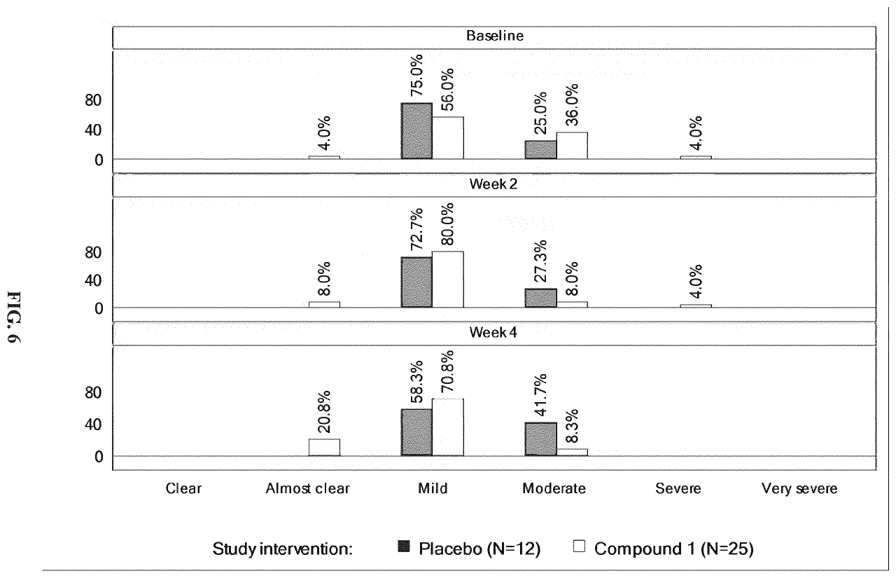

FIG. 6 shows the severity of psoriasis of patients as measured by sIGA score in the treatment arm vs placebo arm at baseline, week 2, and week 4.

Overall, Compound 1 at 200 mg BID showed a clinically important change in sIGA score after 4 weeks of treatment as compared with placebo.

### Conclusions on Clinical Response

While milder forms of the disease usually lead to more subtle therapeutic changes, Compound 1 showed a rapid and statistically significant improvement in psoriasis severity compared to placebo as early as week 2, and the improvement sustained through week 4.

The least square (LS) mean difference in percentage improvement of TLS score between Compound 1 and placebo was 10.77% at week 2 and 17.74% at week 4, indicating an overall improvement in TLS score with Compound 1 compared to placebo. Comparison of the LS mean difference between the treatment arm and placebo arm in percentage improvement of TLS score at week 2 (mild: 20.9%; moderate: 10.9%) and week 4 (mild: 37.0%; moderate: 39.7%) shows improvement in TLS score despite disease severity. The LS mean difference in percentage improvement of total PASI score at week 2 (13.61%) and week 4 (19.38%) also indicates an overall improvement in psoriasis severity with Compound 1 compared to placebo. A higher percentage improvement in PASI score was seen in the Compound 1 group versus placebo at week 2 (mild: 21.2% vs 4.8%, more particularly 21.24% vs 4.80%; moderate: 9.3% vs 3.5%, more particularly 9.31% vs 3.54%) and week 4 (mild: 33.8% vs 14.3%, more particularly 33.83% vs 14.26%; moderate: 33.2% vs 19.9%, more particularly 33.19% vs 19.88%) in both sub-categories.

The treatment arm showed significant improvement from baseline in total PASI score compared to the placebo arm at Week 2 (17.73% vs 4.12%, p=0.005) and Week 4 (35.09% vs 15.71%, p=0.009). A clear improvement in TLS score was observed in the treatment arm versus placebo arm at Week 2 (17.06% vs 6.29%, p=0.032) and Week 4 (38.18% vs 20.44%, p=0.012). At Week 4, the proportion of patients achieving improvement by at least 1 severity level in sIGA was 58.3% in the Compound 1 group versus no effect (0%) in the placebo group. sIGA score also demonstrated how a higher percentage of patients receiving Compound 1 improved from ‘moderate’ disease at baseline, to ‘mild’ or ‘almost clear’ at week 2 and week 4, in comparison to the placebo group. Patients from the placebo group showed an increase in sIGA from ‘mild’ to ‘moderate’.

### Results—Pharmacokinetics

Mean pre-dose and post-dose plasma concentrations of Compound 1 were consistent with analogous values observed at the same dose in a previous study in healthy participants. Mean pre-dose plasma levels of Compound 1 were similar from Day 8 through Day 29, indicating that steady state was achieved and maintained. Plasma concentrations of Compound 1 were below the limit of quantification in the placebo group.

### Results—Adverse Events

Treatment-emergent adverse events (TEAEs) were reported in 7 (58.3%) out of 12 participants in the placebo group and 24 (92.3%) out of 26 participants in the Compound 1 treated group. There were no serious AEs (SAEs), severe TEAEs or AEs of special interest (AESI). There were 2 TEAEs leading to permanent study intervention discontinuation for 2 participants under Compound 1 200 mg BID; one participant dropped out on Day 15 due to nausea assessed by the investigator as drug related, another participant dropped out on Day 8 due to CPK increase (grade 2) assessed by the investigator as non-drug related and linked to increased physical activity, CPK value returned to baseline level after 7 days.

The most frequent TEAEs by primary system organ class (SOC) were nervous systems disorders [4 (33.3%) in placebo and 19 (73.1%) in Compound 1 treated groups], gastrointestinal disorders [2 (16.7%) in placebo and 8 (30.8%) in Compound 1 treated groups] and infections and infestations [4 (33.3%) in placebo and 4 (15.4%) in Compound 1 treated groups]. The most frequent TEAEs by preferred term (PT) were Dysgeusia [0 (0%) in placebo and 16 (61.5%) in Compound 1 treated groups], and headache [4 (33.3%) in placebo and 8 (30.8%) in Compound 1 treated groups]. All TEAEs were of grade 1 or grade 2 severity and participants fully recovered.

There were few potentially clinically significant abnormalities (PCSA) for vital signs, ECG and laboratory parameters scattered across placebo and Compound 1 treated groups. All were considered not clinically meaningful by the investigator. There was no QTcF change from baseline >60 msec and/or QTcF>480 msec. One participant under Compound 1 200 mg BID had QTcF>450 msec (459 msec) on one single occasion on Day 8 with QTcF change of 33 msec from baseline (baseline QTcF value of 426 msec). There was no PCSA for ALT increase, two participants under Compound 1 200 mg BID with total bilirubin increased >1.5 ULN on one single occasion on Day 8 (1.67 ULN, baseline value was 1.5 ULN) and on Day (1.56 ULN, baseline value was 1.3 ULN), respectively.

In summary, no serious adverse events, severe TEAEs or AESI were reported.

### Conclusions on Pharmacodynamics, Pharmacokinetics, and Safety

Compound 1, a specific inhibitor of TNFR1 signalling, showed consistent clinical efficacy in a 4-week treatment period. 200 mg BID of Compound 1 over 28 days was safe and well-tolerated by participants with mild-to-moderate psoriasis, with no serious AEs, severe treatment emergent AEs, or AESI being reported.

The observed Compound 1 plasma concentrations were consistent with pharmacokinetic data previously obtained in healthy participants. Based on mean pre-dose plasma concentrations, steady state was achieved within the first week and maintained until the end of the treatment.

### Results—Analysis of Biomarkers

As indicated above, peripheral blood samples collected at predetermined time points were processed to quantify serum levels of biomarkers of psoriasis including IL-17A, IL-17F, and IL-22. Levels of IL-17A and IL-17F were quantified using a single molecule counting sandwich immunoassay (SMCxPro™, Millipore—see also Hwang et al., Methods (2019) 158:69-76). Levels of IL-22 were quantified using a single-molecule array ELISA (SIMOA™, Myriad RBM—see also Rissin et al., Anal Chem (2011) 83(6):2279-2285). Statistical analysis was based on geometric mean ratio to baseline; a two-sample t-test at significance alpha level of 5% was used for calculation of the p-values. Table 7 below shows changes in biomarker levels summarized using descriptive statistics (mean, standard error of the mean [SEM]) by treatment and time point.

TABLE 7
Change from baseline over time in the levels of systemic biomarkers of psoriasis

| | | | | | Ratio from baseline | |
|---|---|---|---|---|---|---|
| | | Levels of biomarkers | | | Geometric mean | |
| | | Geometric mean (Geometric SE) | | | (Geometric SE) | |
| Biomarker | Treatment | Baseline | Week 2 | Week 4 | Week 2 | Week 4 |
| IL-17A | Compound 1 | 0.62 (1.15) | 0.56 (1.13) | 0.45 (1.14) | 0.92 (1.14) | 0.72 (1.17) |
| (pg/mL) | Placebo | 0.69 (1.16) | 0.89 (1.22) | 0.76 (1.17) | 1.30 (1.12) | 1.10 (1.15) |
| IL-17F | Compound 1 | 2.63 (1.19) | 1.65 (1.21) | 1.35 (1.21) | 0.61 (1.13) | 0.48 (1.12) |
| (pg/mL) | Placebo | 2.74 (1.23) | 2.39 (1.35) | 2.73 (1.29) | 0.92 (1.22) | 1.00 (1.25) |
| IL-22 | Compound 1 | 4.13 (1.19) | 2.86 (1.18) | 2.49 (1.15) | 0.66 (1.10) | 0.57 (1.09) |
| (pg/mL) | Placebo | 2.55 (1.21) | 2.87 (1.18) | 3.06 (1.23) | 1.24 (1.08) | 1.20 (1.12) |

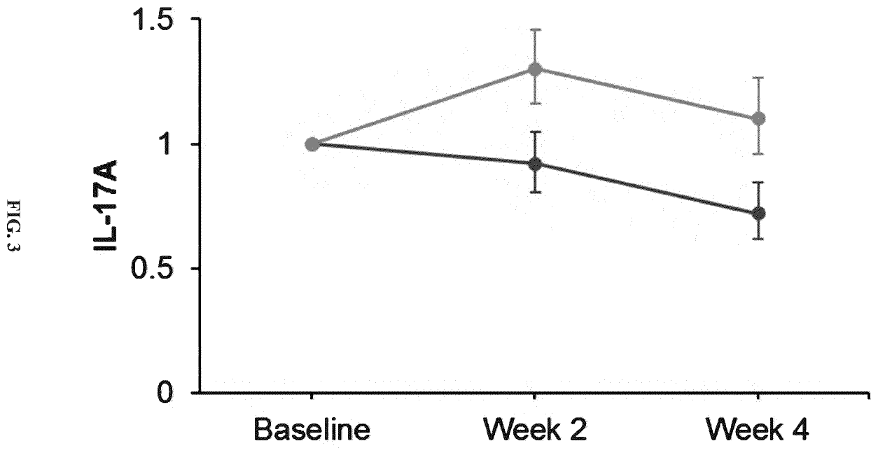

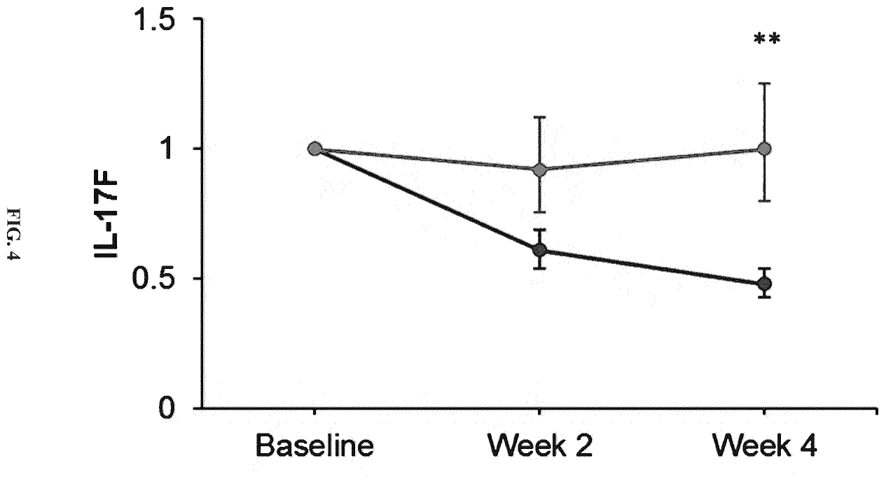

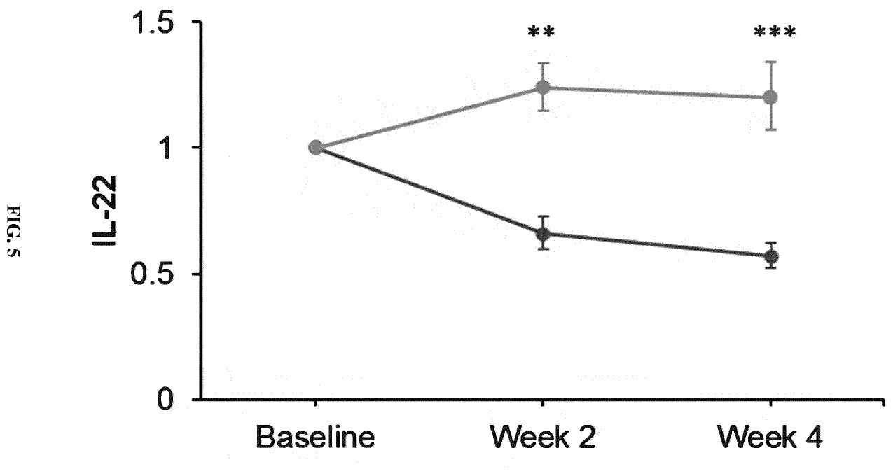

Compared with placebo, Compound 1 achieved rapid and significant reduction in serum IL-22 levels by week 2 (0.66 vs 1.24; p=0.0001) that further decreased at week 4 (0.57 vs 1.20; p<0.0001). IL-17F also declined substantially in treated patients compared with placebo group, both at week 2 (0.61 vs 0.92; p=0.0795) and week 4 (0.48 vs 1.00; p=0.0025). A gradual decline in IL-17A levels from baseline to week 2 (0.92 vs 1.30; p=0.1066) and through week 4 (0.72 vs 1.10; p=0.0859) was observed. The evolution of biomarkers from baseline to week 4 (IL-17A, IL-17F, and IL-22) are shown in FIGS. 3 to 5, respectively. The diminished cytokine secretion correlated to the statistically significant clinical improvement of PASI in patients treated with Compound 1 compared with placebo at week 2 (17.73% versus 4.12%, p=0.005) and at week 4 (35.09% versus 15.71%, p=0.009).

### Conclusion on Biomarker Levels

Serum levels of IL-17A, IL-17F and IL-22 were substantially decreased after treatment with Compound 1 for 4 weeks in patients with mild-to-moderate psoriasis. The biomarker decreases correlated with observed clinical improvement of patients receiving Compound 1.

Overall Conclusions from the Clinical Study

The findings from this phase 1 trial demonstrated that Compound 1, a specific TNFR1 signal inhibitor, was safe, well tolerated and clinically effective, with sustained clinical response, in mild-to-moderate psoriasis over a 4-week treatment period.

It is to be understood that while the disclosure has been described in conjunction with the above embodiments, that the foregoing description and examples are intended to illustrate and not limit the scope of the disclosure. Other aspects, advantages, and modifications within the scope of the disclosure will be apparent to those skilled in the art to which the disclosure pertains.

In addition, where features or aspects are described in terms of Markush groups, those skilled in the art will recognize that such features or aspects are also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A compound for use in a method of treating psoriasis in a human subject, wherein the compound is (7R,14R)-11-[2-(1-aminocyclobutyl)pyrimidin-5-yl]-1-(difluoromethoxy)-6-methyl-6,7-dihydro-7,14-methanobenzimidazo[1,2-b][2,5]benzodiazocin-5(14H)-one (Compound 1): 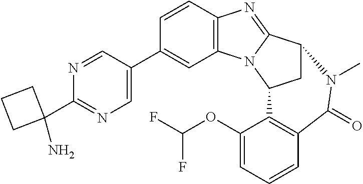 [SMILES: CN1C(=O)c2cccc(OC(F)F)c2[C@H]2C[C@@H]1c1nc3ccc(-c4cnc(C5(N)CCC5)nc4)cc3n12]
or a pharmaceutically acceptable salt thereof, wherein the subject has mild to moderate psoriasis, and wherein the method comprises administering to the subject a daily dose of about 400 mg of the compound (calculated as the free base).

2. The compound for use according to claim 1, wherein the psoriasis is plaque psoriasis, e.g., chronic plaque psoriasis.

3. The compound for use according to claim 1 or claim 2, wherein the severity of psoriasis in the subject is assessed using PASI scoring, and wherein the subject has a total PASI score which is ≤16.

4. The compound for use according to any one of claims 1 to 3, wherein the subject has a total PASI score which is ≥10.

5. The compound for use according to any one of claims 1 to 4, wherein the subject has at least two lesions with TLS score ≥4 (excluding the scalp).

6. The compound for use according to any one of claims 1 to 5, wherein the compound is administered to the subject orally.

7. The compound for use according to any one of claims 1 to 6, wherein the compound is administered in the form of an oral pharmaceutical composition comprising Compound 1, or a pharmaceutically acceptable salt thereof, and at least one pharmaceutically acceptable excipient.

8. The compound for use according to claim 7, wherein the oral pharmaceutical composition is a tablet.

9. The compound for use according to any one of claims 1 to 8, wherein the method comprises administering to the subject a dose of about 200 mg of the compound (calculated as the free base) twice daily.

10. A method of treating psoriasis as defined in any one of claims 1 to 9, wherein a therapeutically effective amount of Compound 1 (or a pharmaceutically acceptable salt thereof) is administered to a human subject in need thereof, and wherein the therapeutically effective amount is a daily dose of about 400 mg (calculated as the free base).

11. Compound 1, or a pharmaceutically acceptable salt thereof, for use in the manufacture of a medicament for treating mild to moderate psoriasis in accordance with a method as defined in any one of claims 1 to 9, wherein the medicament is adapted to provide a daily dose of about 400 mg of the compound (calculated as the free base).
